(19) United States
(12) Patent Application Publication
(10) Pub. No.: US 2005/0096354 A1
(43) Pub. Date: May 5, 2005

(54) Process for the preparation of substituted aryl pyrazoles

(75) Inventors: Iain Robert Gladwell, Sandwich (GB); John George Matthews, Sandwich (GB); Alan John Pettman, Sandwich (GB)

Correspondence Address: SCULLY SCOTT MURPHY & PRESSER, PC, 400 GARDEN CITY PLAZA, SUITE 300, GARDEN CITY, NY, 11530, US

(73) Assignee: Pfizer, Inc., New York, NY (US)

(21) Appl. No.: 10/932,390

(22) Filed: Sep. 2, 2004

Related U.S. Application Data
(60) Provisional application No. 60/517,349, filed on Nov. 4, 2003.
(60) Provisional application No. 60/600,405, filed on Aug. 9, 2004.

(30) Foreign Application Priority Data
Sep. 4, 2003 (GB) 0320719.8
Jul. 2, 2004 (GB) 0414893.8

(51) Int. Cl. C07D 43/02; C07D 231/12; A61K 31/4439; A61K 31/415

(52) U.S. Cl. 514/341; 514/406; 546/275.4; 548/377.1

(57) ABSTRACT

The present invention relates to a process for the preparation of pesticidal compounds, and more particularly to the preparation of pyrazole compounds. In particular, the present invention relates to a process for preparing 1-arylpyrazoles and 1-pyridylpyrazoles which have pesticidal activity. More particularly, the present invention relates to a novel process by which 3,4,5-trisubstituted 1-arylpyrazoles may be produced directly in a reaction which involves coupling of an aryldiazonium species with an appropriately substituted precursor bearing a desired substituent.

### CROSS REFERENCE TO RELATED APPLICATIONS

This application is based upon and claims the benefit of priority from United Kingdom Patent Application No. 0320719.8 filed Sep. 4, 2003, U.S. Patent Application No. 60/517,349 filed Nov. 4, 2003, United Kingdom Patent Application No. 0414893.8 filed Jul. 2, 2004 and U.S. Patent Application No. 60/600,405 filed Aug. 9, 2004, the entire contents which is expressly incorporated herein by its reference.

### FIELD OF THE INVENTION

The present invention relates to a process for the preparation of pesticidal compounds, and more particularly to the preparation of pyrazole compounds. In particular, the present invention relates to a process for preparing 1-phenylpyrazoles and 1-pyridylpyrazoles, hereinafter referred to as 1-arylpyrazoles.

### BACKGROUND OF THE INVENTION

Arylpyrazoles are described widely in the prior art and European Patent Publication Nos EP 0295117, EP 0234119, EP 0946515, EP 0871617, EP 0846686 and EP 0918756 describe many such compounds.

Certain pyrazole derivatives possessing, inter alia, antiparasitic activity are already known. For example, EP-A-0234119 discloses 1-arylpyrazoles for the control of arthropod, plant nematode and helminth pests. 1-arylpyrazoles are also disclosed in EP-A-0295117; in addition to having arthropodicidal, plant nematocidal and antihelminthic activity, these compounds are reported to display antiprotozoal properties. Similar profiles of activity are also displayed by the 1-arylpyrazoles disclosed in EP-A-0295118.

The prior art in general has previously described a reaction for forming 1-arylpyrazoles which is known as the Japp-Klingemann reaction. This reaction is well known in the chemical literature and is described in Org. React., 1959, 10, 143-178. In this reaction, an aryl diazonium species is reacted with a tri-substituted methane derivative in which two of the substituents are electron withdrawing groups. If the third substituent is, like the fourth substituent, hydrogen then a hydrazone is formed. In the case in which the third substituent is a group such as a methylene nitrile then cyclisation occurs to produce an arylpyrazole bearing an electron withdrawing group at the C-3 position, an amine at the C-5 position with the C-4 position being unsubstituted.

J Prakt Chem 1989, 331 describes the reaction of phenacylmalononitriles with hydrazine or phenylhydrazine to produce phenacylpyrazole derivatives, and the reaction of phenacylmalononitriles with diazonium cations to form aminopyrazole derivatives. However, the reaction suffers the disadvantage that it takes over 24 hours to complete and during that time the temperature must be maintained below room temperature. The reaction also requires recrystallisation of the product after isolation in order to return a product of reasonable purity.

WO98/40358 describes a process for preparing pyrazole derivatives in which an aryl diazonium derivative is cyclised to form the pyrazole ring. In this process, the leaving group which is normally lost in this type of reaction is re-incorporated into the resulting pyrazole ring at the carbon 4-position having been lost from the carbon which forms the 3-position of the pyrazole ring. The advantage of this process is said to be that it gives access to 3,4,5-trisubstituted-1-aryl pyrazoles. Thus, the substituent which was originally present at the C-3 position migrates to the C-4 position during the cyclisation rather than acting as a leaving group.

However, a significant disadvantage of the process of WO 98/40358 is that the group installed at the 4-position of the pyrazole ring is constrained by the chemistry to be electron-withdrawing, e.g. alkoxycarbonyl. It is therefore necessary to perform further synthetic steps to form 3,4,5-trisubstituted pyrazoles with more varied 4-substituents. Futhermore, the groups that can be introduced in this way are limited to those derivable from the 4-substituent originally introduced.

A further method for preparing 4-substitued pyrazoles relies on further transformations of a 3,5-disubstituted-4-[H]-pyrazole, e.g. by introduction of an iodo substituent, and further synthetic steps. Both of the above strategies suffer from the disadvantages of long, non-linear synthetic sequences and lack of versatility in the array of 4-substituents that can be thus introduced.

EP 888291 discloses a process for preparing 2,3-dicyanopropionate derivatives by reacting a cyanoacetate with cyanide salt and formaldehyde or a source of formaldehyde. The 2,3-dicyanopropionate product is then reacted with a diazonium salt to produce a 1-aryl pyrazole compound. However, the resulting 1-arylpyrazole is unsubstituted at the 4-position and thus further reactions are needed to produce 4-substituted derivatives. This in turn leads to additional waste, additional time and reduced yield and purity of the 4-substituted product.

### SUMMARY OF THE INVENTION

It is an aim of the present invention to overcome the various disadvantages associated with prior art processes. Thus it is an aim of the invention to produce a 1-arylpyrazole in a convenient reaction which does not require a large amount of maintenance by laboratory personnel and which can be completed in a relatively short time. It is thus an aim of the present invention to provide a synthetically efficient process for the production of pyrazole derivatives which allows access to compounds not readily accessible using existing art, and which avoids the problems of either having to leave the reaction for an extended period of time or of having to control carefully the conditions so that the temperature does not rise or fall too much during the period of the reaction so the reaction fails. It is also an aim to provide a process in which the convergency (ie the bringing together of synthetic fragments) is maximised. It is thus an aim to provide a route to the compounds of formula (I) which offers an improved yield relative to the existing routes. It is a further aim of the process of the present invention to avoid the use of unnecessary synthetic steps and/or purification steps. It is a further aim of the present invention to provide a process which minimizes the number of synthetic steps required and which avoids the problem of competing reactions and/or the disposal of hazardous materials. It is also an aim to provide a route which gives access to a range of 3, 4 and 5-substituted aryl pyrazoles.

We have found a novel process by which 3,4,5-trisubstituted 1-arylpyrazoles may be produced directly in a reaction which involves coupling of an aryldiazonium species with an appropriately substituted precursor bearing a desired substituent. The desired substituent is introduced concomitantly at the C-4 position in a process which does not involve any rearrangement. Furthermore, the reaction produces the tri-substitued pyrazole directly. This removes the need for a lengthy synthetic procedure and the need for several work-ups of the intermediate products and results in good yields. The process of the present invention has the significant advantage that the C-4 substituent may be built into the original tetrasubstituted ethane derivative which is one of the starting materials and which is reacted with the aryldiazonium species to form the pyrazole. Control of the position of substitution on the resulting pyrazole ring is therefore absolute in the reaction of the present invention. Furthermore, a very wide variety of 4-substituents may be introduced conveniently and directly.

The process of the invention has a significant advantage relative to WO98/40358 in that the 3,4,5-trisubstituted aryl pyrazole may be obtained in a single reaction without the need for further synthetic procedures.

### DETAILED DESCRIPTION OF THE INVENTION

The present invention thus provides a process for the preparation of a compound of formula (I) 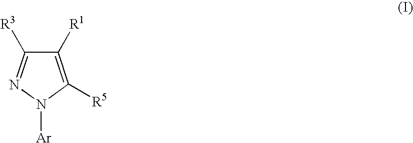
said process comprising the step of reacting a compound of formula (II) 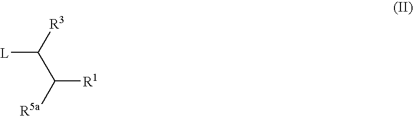
with a compound of formula (III)
Ar—N≡N⁺X⁻ (III)
optionally in the presence of an acid, wherein:
Ar is phenyl or pyridyl, optionally independently substituted by 1 to 4 groups selected from the group comprising: C₁₋₄ alkyl, C₁₋₄ alkoxy, C₁₋₄ alkylthio, C₁₋₄ alkylsulphinyl, and C₁₋₄ alkylsulphonyl, wherein each of these optional substituent groups may itself be substituted by one or more halogen atoms selected independently; pentafluorosulfur; and —COOC₁₋₈ alkyl;
R¹ is C₁₋₈ alkyl, C₂₋₈ alkenyl provided that said alkenyl is not conjugated with the double bond shown in formula (IV), C₄₋₈ cycloalkyl, C₁₋₈ alkyl(C₃₋₈ cycloalkyl), a 5- or 6-membered heterocycle which may be saturated, partially or fully unsaturated designated ‘het’ containing 1, 2 or 3 heteroatoms, which are independently selected from 1, 2, or 3 N atoms, 1 or 2 O atoms and 1 or 2 S atoms, where the valence allows, C₁₋₈ alkylhet, phenyl, C₁₋₈ alkylphenyl; wherein each of the preceding groups may be optionally independently substituted by 1 to 4 groups selected from the group comprising: halogen, C₁₋₄ alkyl, C₁₋₄ alkoxy, C₁₋₄alkylthio, and —COOC₁₋₈ alkyl; wherein each of these preceding optional substituent groups may be substituted where possible by one or more halogen atoms selected independently; or
R¹ is a group of formula (A): 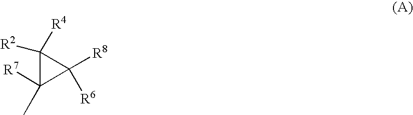
   wherein R² and R⁴ are each independently selected from hydrogen, C₁₋₄ alkyl, fluoro, chloro and bromo, or, together with the carbon atom to which they are attached, form a C₃₋₆ cycloalkyl group;
R⁶ and R⁸ are each independently selected from hydrogen, C₁₋₄ alkyl, fluoro, chloro and bromo;
or when R² and R⁴ do not form part of a cycloalkyl group, R² and R⁶, together with the carbon atoms to which they are attached, may form a C₅₋₇ cycloalkyl group;
R⁷ is hydrogen, C₁₋₄ alkyl optionally substituted with one or more halo, or C₁₋₄ alkoxy;
or R¹ is a fused bicyclic moiety “AB” where the “A” ring is as defined as ‘het’ above and the “B” ring fused thereto in “AB” is a 5- or 6-membered saturated or partially or fully unsaturated carbocycle, or saturated or partially or fully unsaturated heterocycle where the valence allows, which heterocycle contains 1, 2, 3 or 4 hetero-atoms independently selected from 1, 2, 3 or 4 N atoms, 1 or 2 O atoms and 1 or 2 S atoms, where the valence allows,
said R¹ group being linked via the “A” ring to the 4-position of the pyrazole via a carbon-carbon bond,
and said R¹ group being optionally substituted by one or more substituents independently selected from halogen, C₁₋₆ alkyl optionally substituted by one or more halogen atoms, C₁₋₆ alkoxy optionally substituted by one or more halogen atoms, C₁₋₆ alkoxycarbonyl optionally substituted by one or more halogen atoms, NO₂, NH₂, CN or S(O)ₘ(C₁₋₄ alkyl optionally substituted by one or more halogen atoms) where m is 0, 1 or 2;
R³ is selected from the group comprising: CN, CF₃, CHO, COR and COOR wherein R is C₁₋₆ alkyl optionally substituted by one or more halogen atoms which may be the same or different;
R⁵ is selected from the group comprising: hydrogen, C₁₋₆ alkyl optionally substituted by one or more halogen atoms which may be the same or different, OH and NH₂;
R^{5a} is selected from the group comprising: CN, COOH, CHO, COR and COOR wherein R is C₁₋₄ alkyl optionally substituted by one or more halogen atoms which may be the same or different;
L is an activating group; and
X— is a compatible counter ion,
followed by removal of group L.

The counter ion X⁻ may be any suitable counter ion normally found in diazonium reactions. Preferably, X⁻ is halogen, HSO₄⁻, or tetrafluoroborate and most preferably is tetrafluoroborate.

The group L is an electron withdrawing group which stabilises the anion intermediate in the process. Thus preferably L is a group which is capable of stabilising a negative charge on an adjacent carbon atom. The group L must also be removable. L can be removed under basic conditions, for example by base hydrolysis or can be removed by reduction and/or elimination. The group L is important as it serves to direct the reaction of the diazonium species with the compound of formula (II) but then is removed in the subsequent stages of the reaction.

Preferably L is an ester group or a group COR¹⁰. More preferably, L is a group selected from: —S(O)ₚR⁹ where p is 1 or 2, (R⁹O)₂PO, COOR⁹ and —COR¹⁰,

wherein R⁹ is selected from: C₁₋₈ alkyl, C₃₋₈ cycloalkyl, (CH₂)ₙPh and (CH₂)ₙ heteroaryl wherein n=0, 1 or 2, each of which groups may be optionally substituted on any carbon atom by one or more groups selected independently from: halogen, hydroxy, cyano, nitro, C₁₋₄ alkoxy, C₁₋₄ haloalkoxy, C₁₋₄ alkanoyl, C₁₋₄ haloalkanoyl, C₁₋₄ alkylsulphinyl, C₁₋₄ haloalkylsulphinyl, C₁₋₄ alkylsulphonyl, C₁₋₄ haloalkylsulphonyl, C₁₋₄ cycloalkyl and C₃₋₈ halocycloalkyl; and R⁹ can be hydrogen; and

wherein R¹⁰ is selected from: C₁₋₈ alkyl, di-C₁₋₈ alkylamino, C₁₋₈ alkylthio, C₃₋₈ cycloalkyl, (CH₂)ₙPh and (CH₂)ₙ heteroaryl wherein n=0, 1 or 2, each of which groups may be optionally substituted on any carbon atom by one or more groups selected independently from: halogen, hydroxy, cyano, nitro, C₁₋₄ alkoxy, C₁₋₄ haloalkoxy, C₁₋₄ alkanoyl, C₁₋₄ haloalkanoyl, C₁₋₄ alkylsulphinyl, C₁₋₄ haloalkylsulphinyl, C₁₋₄ alkylsulphonyl, C₁₋₄ haloalkylsulphonyl, C₃₋₈ cycloalkyl and C₃₋₈ halocycloalkyl; and R¹⁰ can be hydrogen.

Preferably L is a group selected from COOR⁹ and COR¹⁰.

More preferably, L is a group selected from: —COOC₁₋₈ alkyl, —COOPh and —COOCH₂Ph, each being optionally substituted by one or more groups independently selected from: halogen, hydroxy, C₁₋₄ alkoxy, and C₁₋₄ haloalkoxy; and —COOH.

More preferably, L is —COOC₁₋₈ alkyl, optionally substituted by one or more groups independently selected from: halogen, hydroxy, C₁₋₄ alkoxy, and C₁₋₄ haloalkoxy.

Most preferably L is —COOMe or —COOEt.

In certain cases, the nature of the leaving group L means that the resulting intermediate is in the wrong oxidation state. Thus, where necessary, one or more reaction steps may be added to ensure the correct oxidation state is reached prior to cyclising to form the aryl pyrazole. For example, where L is a sulphonyl group it may be necessary to perform a reduction step with a conventional reducing agent such as sodium amalgam to bring the resulting intermediate into the correct oxidation state for subsequent cyclisation to the aryl pyrazole. Alternatively, the sulphonyl or sulphinyl group may be eliminated using a base such as DBU (1,8-diazabicyclo[5.4.0]undec-7-ene) followed by reduction with a complex metal hydride such as sodium borohydride.

The process has a number of embodiments which are preferred because the reaction works well or because the end product of the process is of particular utility.

Preferably, the Ar group is tri-substituted, and more preferably it is substituted at the 2-, 4-, and 6-positions with an optional substituent selected from the group comprising: halogen, C₁₋₄ alkyl, C₁₋₄ alkoxy, C₁₋₄ alkylthio, SF₅ and —COOC₁₋₈ alkyl, wherein each of these optional substituent groups may itself be substituted where chemically possible by one to three halogen atoms selected independently.

It is further preferred that Ar is phenyl.

More preferably, Ar is a phenyl group which bears substituents at the 2-, 4-, and 6-positions, the substituents at those positions being independently selected from chloro, trifluoromethyl, trifluoromethoxy, and pentafluorosulfur.

Preferably, R¹ is selected from: C₁₋₈ alkyl, C₄₋₈ cycloalkyl, a group of formula (A) where A is as defined above, a 5- or 6-membered heterocycle which may be saturated or unsaturated designated ‘het’, C₁₋₈ alkylhet, phenyl, and C₁₋₈ alkylphenyl, wherein each of the preceding groups may be optionally independently substituted by 1 to 4 groups selected from the group comprising: halogen, C₁₋₄ alkyl, C₁₋₄ alkoxy, C₁₋₄ thioalkoxy, and —COOC₁₋₈ alkyl, wherein each of these optional substituent groups may itself be substituted where possible by one or more halogen atoms selected independently.

More preferably, R¹ is C₁₋₈ alkyl, C₄₋₈ cycloalkyl, a group of formula (A) where A is as defined above, or a 5- or 6-membered heterocycle which may be saturated or unsaturated designated ‘het’, or C₁₋₈alkylhet.

Het is preferably selected from pyrazolyl, imidazolyl, oxazolyl, isoxazolyl, thiazolyl, isothiazolyl, furanyl, thiophenyl, pyrrolyl, and pyridyl wherein the aforementioned groups may be optionally substituted by 1, 2 or 3 halogen atoms. More preferably, het is selected from pyrazolyl and isoxazolyl; most preferably het is selected from pyrazol-4-yl, oxazol-3-yl and oxazol-4-yl. Thus when R¹ is het it is most preferred that it is selected from pyrazolyl and isoxazolyl; and in particular from pyrazol-4-yl, oxazol-3-yl and oxazol-4-yl.

It is further preferred that R¹ is selected from C₃₋₈ cycloalkyl and het, and more preferably R¹ is C₃₋₈ cycloalkyl and is most preferably selected from cyclopropyl and cyclobutyl. Most preferably, R1 is a group of formula (A).

Thus, a preferred group of compounds of formula (I) that can be made by the process of the present invention are those wherein:
R¹ is a group of formula (A);
Ar is 2,6-dichloro-4-trifluoromethylphenyl, 2,6-dichloro-4-pentafluorothiophenyl, 2,4,6-trichlorophenyl or 3-chloro-5-trifluoromethylpyridin-2-yl;
R³ is cyano, trifluoromethyl, formyl, or acetyl;
R² and R⁴ are each independently selected from hydrogen, methyl, fluoro, chloro and bromo or, together with the carbon atom to which they are attached, form a cyclopropyl, cyclobutyl or cyclopentyl group;
R⁶ and R⁸ are each independently selected from hydrogen, methyl, chloro and bromo;
or, when R² and R⁴ do not form part of a cycloalkyl group, R² and R⁶, together with the carbon atoms to which they are attached, may form a cyclopentane or cyclohexane group; and R⁷ is hydrogen, methyl, ethyl, trifluoromethyl, chlorodifluoromethyl, pentafluoroethyl, heptafluoropropyl or methoxy.

Preferably, R³ is cyano.

Preferably, R⁵ is amino.

Thus, a more preferred group of compounds of formula (I) that can be produced is that wherein:
R¹ is a group of formula (A);
Ar is 2,6-dichloro-4-trifluoromethylphenyl, or 2,6-dichloro-4-pentafluorothiophenyl;
R³ is cyano;
R⁵ is amino;
R² and R⁴ are the same and are hydrogen, chloro or bromo;
R⁶ and R⁸ are hydrogen; and
R⁷ is hydrogen, trifluoromethyl or chlorodifluoromethyl.

In the above definitions, halo means chloro, fluoro, bromo, or iodo. Alkyl and alkoxy groups containing the requisite number of carbon atoms can be unbranched- or branched-chain.

Particularly preferred individual compounds that can be made by the process of the invention include:
5-amino-3-cyano-4-(2,2-dibromocyclopropyl)-1-(2,6-dichloro-4-trifluoromethylphenyl)pyrazole;
5-amino-3-cyano-4-(2,2-dibromocyclopropyl)-1-(2,6-dichloro-4-pentafluorothiophenyl)pyrazole;
5-amino-3-cyano-4-(2,2-dichlorocyclopropyl)-1-(2,6-dichloro-4-pentafluorothiophenyl)pyrazole; and
5-amino-3-cyano-1-(2,6-dichloro-4-trifluoromethylphenyl)-4-(1-trifluoromethylcyclopropyl)pyrazole.

The process is most advantageously used to prepare 5-amino-3-cyano-1-(2,6-dichloro-4-trifluoromethylphenyl)-4-(1-trifluoromethylcyclopropyl)pyrazole as described in the Example Preparation below.

Ideally, the solvent should be a polar solvent which does not react with either the diazonium salt or cation, or with the compound of formula (II). Suitable solvents include individual solvents or a mixture of solvents selected from: alcohols such as methanol, ethanol and propanol, acetonitrile, dimethylformamide, dimethylsulfoxide, ethers such as diethyl ether and tetrahydrofuran, halogenated solvents such as dichloromethane, pyridine and water. Preferred solvents include: methanol, acetonitrile, dichloromethane, pyridine and water or a mixture including at least two of these.

The reaction may optionally be carried out under mildly acidic conditions. Suitable acids include: sulphuric acid, hydrochloric acid, glacial acetic acid, and tetrafluoroboric acid. The pH of the reaction mixture may be increased after reaction by addition of a base to facilitate the removal of the leaving group L. Suitable bases include: hydroxides and carbonates of alkali metals, ammonium hydroxide, or organic bases such as pyridine. The product may be recovered after reaction by conventional workup procedures. Ideally, the product is recovered by solvent extraction or evaporation of the solvent. The product may be further purified as necessary by column chromatography or by recrystallisation.

The diazonium salt of formula (III) can be produced by conventional means and may be prepared in situ for further reaction or can be isolated and used in a subsequent reaction step. For example, treatment of an arylamine in a suitable solvent such as ethanol or water with a nitrite ion such as sodium nitrite or isoamyl nitrite in the presence of a strong acid such as tetrafluoroboric acid or sulphuric acid with optional diethyl ether, at temperatures between −5-60° C. produces a diazonium salt (III) which can either be isolated by filtration or treated with a dicyano compound of formula (II).

Coupling of the in situ generated aryldiazonium cation with the dicyano compound of formula (II) is achieved by stirring in a suitable solvent such as water, methanol, dichloromethane, and/or acetonitrile, and treatment with a cosolvent such as acetic acid, followed by addition of a suitable base such as ammonia solution and/or ammonium hydroxide at room temperature for 1-24 hours.

Alternatively, coupling of the isolated aryldiazonium cation with the dicyano compound of formula (II) is achieved by stirring in a suitable solvent such as methanol, dichloromethane, water and/or acetonitrile, and treatment with an optional cosolvent such as acetic acid, followed by treatment with base at room temperature for 1-24 hours. Suitable base additives include N,N-dimethylaminopyridine, sodium acetate, sodium carbonate, sodium hydrogen carbonate, ammonia solution, ammonium hydroxide and/or pyridine.

In another aspect, the invention provides a process for the preparation of a compound of formula (II) in which R^{5a} is CN 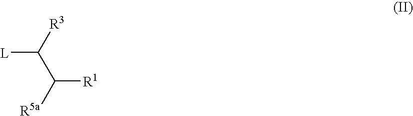
the process comprising treating a compound of formula (IV) 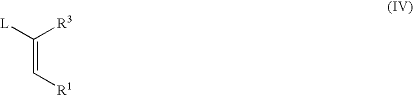
with a source of cyanide ions, wherein L, R¹, and R³ are as defined above.

The compound of formula (IV) can be dissolved in a polar solvent to which an aqueous or alcoholic cyanide salt is added. The cyanide salt is an inorganic salt and is preferably an alkali metal cyanide, with sodium or potassium cyanide being preferred.

For example, reaction of a compound of formula (IV) with potassium cyanide in a polar solvent such as methanol at temperatures between 0-30° C. for a few hours followed by optional addition of a mild acid such as acetic acid produces a compound of formula (II).

In another aspect, the invention provides a process for the preparation of a compound of formula (II) in which R^{5a} is CN, COOH, CHO, COR, and COOR wherein L, R¹ and R³ are as defined above in relation to compounds of formula (I) 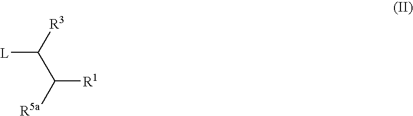
the process comprising reacting a compound of formula LCH₂R³ with a base and then reacting the resulting mixture with R^{5a}CH(X)R¹ where X is Cl, Br, I, C₁₋₈ alkylsulphonate or arylsulphonate at room temperature under an inert atmosphere, for example under a nitrogen atmosphere.

Preferably, the base is a metal hydride, such as sodium hydride, or an alkoxide, such as sodium methoxide. Any suitable solvent may be used for this reaction. Preferably, the solvent is DMF or an alcohol such as methanol, ethanol or propanol, or is an ether such as diethyl ether or tetrahydrofuran.

References for the processes to prepare compounds of formula (II) when R^{5a}═CN, CO₂R, COR, CHO and COOH include: WO2000035871; JP2002249476;
Sharygin et al, Khimicheskoe Mashinostroenie i Teckhnologiya, 1986, 23, 17-20;
Chakravarti et al, Bulletin of the Calcutta School of Tropical Medicine, 1966, 14, 1, 15; and
Larcheveque et al, Synthesis, 1991, 2, 162.

In certain circumstances, the reaction to produce the compound of formula (I) can be carried out in a single step from a compound of formula (II) without isolating the compound of formula (III). The compound of formula (II) is produced from the compound of formula (IV) in situ by reaction of the compound of formula (IV) with a source of cyanide ions.

Thus in another aspect the present invention provides a process for preparing a compound of formula (I) in which R⁵ is NH₂ 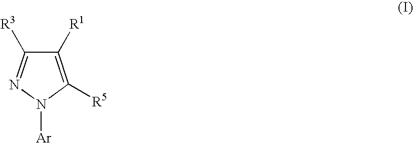
said process comprising a first step of reacting a compound of formula (IV) 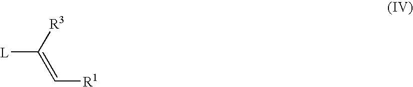
with a source of cyanide ions to produce a compound of formula (II) 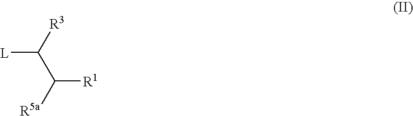
and subsequently treating the reaction mixture with a compound of formula (III)
Ar—N≡N⁺X⁻ (III)
wherein Ar, L, R¹, R³, and X⁻ are as defined above in relation to the compounds of formula (I) and R^{5a} is CN. The compound of formula (II) is isolated after workup and then is reacted with a compound of formula (III) which may be formed in a separate step and added to the reaction or may be formed in situ from a suitable precursor.

In another aspect of the invention, the present invention provides a process for preparing a compound of formula (IV), 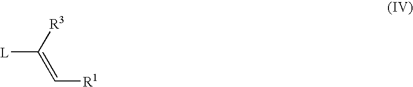
wherein L, R¹ and R³ are as defined in relation to the compounds of formula (I), the process comprising the step of reducing a compound of formula (V) 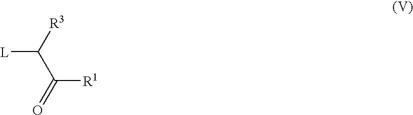
with a complex metal hydride in the presence of acid, wherein L, R¹ and R³ are as defined above in relation to formula (I).

For example, a compound of formula (V) in a mild acidic solvent such as acetic acid may be treated with a selective reducing agent such as sodium triacetoxyborohydride to produce a compound of formula (IV).

Compounds of formula (V) in which L is COOR and R³ is CN can, for example, be made by condensation of methyl cyanoacetate with an acid chloride in an aprotic solvent such as dichloromethane in the presence of a Lewis acid, such as magnesium chloride and a mild base, such as triethylamine. The reaction is carried out at low temperature, preferably between −78 and 0° C.

Acid chlorides can be made using conventional methods, for example by reaction of the corresponding carboxylic acid with oxalyl chloride.

In an alternative embodiment, compounds of formula (IV) may also be synthesised under Knoevenagel conditions by condensation of an alkyl cyanoacetate such as methyl cyanoacetate and a suitable aldehyde, R¹CHO at ambient temperature in the presence of a mild base such as triethylamine or piperidine and solvent such as dichloromethane or acetic acid.

R¹CHO may be obtained according to conventional procedures.

When R¹ is a group of formula (A), the following sequence may optionally be utilised.

Cyclopropanation of an □□-unsaturated ester (VI), in the presence of a hydride donor such as sodium hydride, in a polar solvent such as dimethyl sulfoxide and a suitable carbene source such as trimethylsulfoxonium iodide provides an ester of formula (VII).

Reduction of (VII) with a reducing agent such as a complex hydride, preferably lithium aluminium hydride, provides the alcohol (VIII) which can then be oxidised with a suitable agent such as pyridinium chlorochromate in a suitable aprotic solvent such as dichloromethane to yield the desired aldehyde (IX).

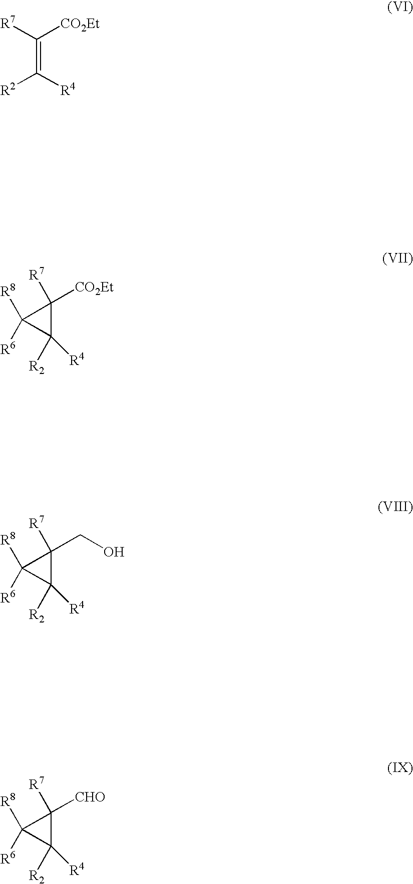

The compounds of formula (II) are novel compounds. In another aspect, the invention provides a compound of formula (II) 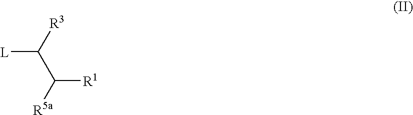
wherein L, R¹, R³ and R^{5a} are as defined above in relation to the compounds of formula (I).

Certain of the compounds of formula (IV) are novel and in another aspect, the invention thus provides a compound of formula (IV) 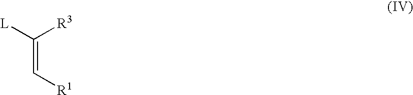
wherein L, R¹ and R³ are as defined above in relation to the compounds of formula (I).

Certain of the compounds of formula (V) are novel and in another aspect, the invention thus provides a compound of formula (V) 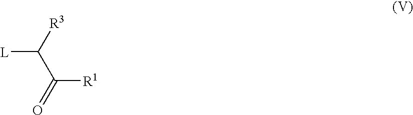
wherein L, R¹ and R³ are as defined above in relation to the compounds of formula (I).

Certain of the compounds of formula (IX) are novel and in another aspect, the invention thus provides a compound of formula (IX) 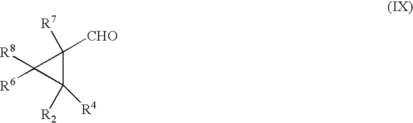
wherein R², R⁴, R⁶, R⁷ and R⁸ is as defined above in relation to the compounds of formula (I).

In the case of the novel compounds of formulae (II), ((V), (V) and (IX), the identities of the preferred and most preferred substituents in each case is the same, and corresponds directly with, the preferred and most preferred substituents defined in relation to the process of the present invention for preparing compounds of formula (I).

The skilled man will appreciate that the compounds of the invention could be made by methods other than those herein described, by adaptation of the methods herein described and/or adaptation of methods known in the art, for example the art described herein, or using standard textbooks such as
“Comprehensive Organic Transformations—A Guide to Functional Group Transformations”, R C Larock, Wiley-VCH (1999 or later editions),
“March's Advanced Organic Chemistry—Reactions, Mechanisms and Structure”, M B Smith, J. March, Wiley, (5th edition or later)
“Advanced Organic Chemistry, Part B, Reactions and Synthesis”, F A Carey, R J Sundberg, Kluwer Academic/Plenum Publications, (2001 or later editions),
“Organic Synthesis—The Disconnection Approach”, S Warren (Wiley), (1982 or later editions),
“Designing Organic Syntheses” S Warren (Wiley) (1983 or later editions), “Guidebook To Organic Synthesis” R K Mackie and D M Smith (Longman) (1982 or later editions), etc.,
   and the references therein as a guide.

It is to be understood that the synthetic transformation methods mentioned herein are exemplary only and they may be carried out in various different sequences in order that the desired compounds can be efficiently assembled. The skilled chemist will exercise his judgement and skill as to the most efficient sequence of reactions for synthesis of a given target compound. For example, substituents may be added to and/or chemical transformations performed upon, different intermediates to those mentioned hereinafter in conjunction with a particular reaction. This will depend inter alia on factors such as the nature of other functional groups present in a particular substrate, the availability of key intermediates and the protecting group strategy (if any) to be adopted. Clearly, the type of chemistry involved will influence the choice of reagent that is used in the said synthetic steps, the need, and type, of protecting groups that are employed, and the sequence for accomplishing the synthesis. The procedures may be adapted as appropriate to the reactants, reagents and other reaction parameters in a manner that will be evident to the skilled person by reference to standard textbooks and to the examples provided hereinafter.

It will be apparent to those skilled in the art that sensitive functional groups may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and refernces therein.

Instruments Used to Acquire Characterising Data

Nuclear magnetic resonance (NMR) spectral data were obtained using Varian Inova 300, Varian Inova 400, Varian Mercury 400, Varian Unityplus 400, Bruker AC 300 MHz, Bruker AM 250 MHz, or Varian T60 MHz spectrometers, the observed chemical shifts (δ) being consistent with the proposed structures. Mass spectral (MS) data were obtained on a Finnigan Masslab Navigator, a Fisons Instruments Trio 1000, or a Hewlett Packard GCMS system model 5971 spectrometer. The calculated and observed ions quoted refer to the isotopic composition of lowest mass. HPLC means high performance liquid chromatography. Room temperature means 20 to 25° C.

Preparations

The following Preparations illustrate the synthesis of certain intermediates used in the preparation of the Examples below.

Preparation 1

### Methyl 2,3-dicyano-3-[1-(trifluoromethyl)cyclopropyl]propanoate

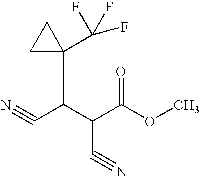

To a solution of Preparation 7 (73.0 mg, 0.33 mmol) in methanol (0.43 ml) was added potassium cyanide (0.02 g, 0.33 mmol) and the reaction mixture was stirred at room temperature for 2 h before concentrating in vacuo to give Preparation 1 (74 mg).

Alternative Synthesis

To a solution of Preparation 7 (100 mg, 0.46 mmol) in methanol (2 ml) at 0° C. was added potassium cyanide (35 mg, 0.55 mmol). The reaction mixture was then stirred for 1 h before silica (2 g) and acetic acid (54.7 mg, 0.91 mmol) were added and the mixture was concentrated in vacuo. The residue was loaded on to an Isolute™ cartridge (silica, 10 g) and purified with gradient elution, pentane:diethyl ether [1:0 to 1:1]. The appropriate fractions were combined and concentrated to give Preparation 1 (60 mg, 0.24 mmol) as a colourless oil.

Preparation 2

### Methyl 2,3-dicyano-3-[2-(trifluoromethyl)cyclopropyl]propanoate

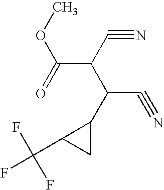

To a solution of Preparation 8 (524 mg, 2.39 mmol) in methanol (10 ml) at 0° C. was added potassium cyanide (187 mg, 2.87 mmol). The mixture was stirred at 0° C. for 1 h and at room temperature for 30 min, before silica was added, followed by acetic acid (215 mg, 3.59 mmol) in methanol (0.5 ml). The reaction mixture was concentrated in vacuo. The crude material was purified by column chromatography using an Isolute™ cartridge (silica, 10 g) and gradient elution, diethyl ether:pentane [1:1 to 2:1]. The appropriate fractions were concentrated to give Preparation 2 (526 mg, 2.14 mmol) as an oil.

Preparation 3

### Methyl 2,3-dicyano-3-(1-methylcyclopropyl)propanoate

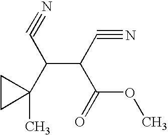

To a solution of Preparation 9 (1 g, 6.09 mmol) in methanol (25 ml) at room temperature was added potassium cyanide (475 mg, 7.31 mmol) and the resulting mixture was stirred for 2 h. To the reaction mixture was added silica and the solution was dried, transferred to a flash chromatography column, and purified with gradient elution, ethyl acetate:cyclohexane [1:10 to 1:0]. The appropriate fractions were concentrated to give Preparation 3 (705 mg, 3.67 mmol, 60%). NMR (CDCl3, selected data): 0.6 (m, 2H), 0.85 (m, 1H), 1.3 (s, 3H), 2.65 (d, 1H), 3.0 (d, 1H), 3.85 (m, 1H), 3.9 (s, 3H).

Preparation 4

### 6-Chloropyridine-3-diazonium Tetrafluoroborate

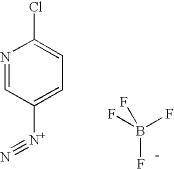

To a stirred solution of 5-amino-2-chloropyridine (655 mg, 5.09 mmol) in ethanol (2 ml) at −5° C. was added tetrafluoroboric acid (8M in water, 1.34 ml, 10.69 mmol). Isoamyl nitrite (0.74 ml, 5.34 mmol) was added dropwise and the reaction mixture was stirred for 30 min at −5° C. The reaction mixture was filtered and the precipitate washed with absolute ethanol and diethyl ether to give Preparation 4 (960 mg, 4.22 mmol, 83%) as a pale yellow solid. NMR (CD3OD, selected data): 7.25 (d, 1H), 8.65 (dd, 1H) 9.5 (s, 1H).

Preparation 5

### 2-Chloropyridine-3-diazonium Tetrafluoroborate

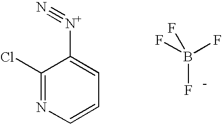

To a stirred solution of 3-amino-2-chloropyridine (655 mg, 5.09 mmol) in ethanol (2 ml) at −5° C. was added tetrafluoroboric acid (8M in water, 1.34 ml, 10.69 mmol). Isoamyl nitrite (0.74 ml, 5.34 mmol) was added dropwise and the reaction mixture was stirred for 30 min at room temperature.

The reaction mixture was filtered and the precipitate washed with absolute ethanol and diethyl ether to give Preparation 5 (988 mg, 4.35 mmol, 86%) as a white/pink solid. NMR (CD3OD, selected data): 7.55 (m, 1H), 8.85 (m, 1H), 8.95 (m, 1H).

Preparation 6

### Ethyl 2,3-dicyano-3-cyclopropylpropanoate

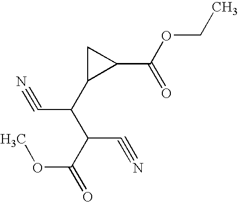

To a solution of Preparation 11 (1.71 g, 7.62 mmol) in methanol (50 ml) at 0° C. was added potassium cyanide (0.59 g, 9.14 mmol) and the reaction mixture was stirred overnight.

Silica and acetic acid (7.62 mmol) were added and the solution was concentrated in vacuo. The crude product was loaded on to an Isolute™ cartridge (50 g) and eluted with pentane/diethyl ether [1:0 to 1:1]. The appropriate fractions were combined and concentrated to give Preparation 6 (400 mg, 1.6 mmol) as a yellow oil. NMR (CDCl3, selected data): 1.25 (m, 3H), 1.45, (m, 1H), 1.75 (m, 1H), 1.95 (m, 1H), (3.25 m, 1H), 3.75 (m, 1H), 3.9 (s, 3H), 3.95 (m, 1H), 4.15 (m, 2H).

Preparation 7

### Methyl(2)-2-cyano-3-[1-(trifluoromethyl)cyclopropyl]acrylate

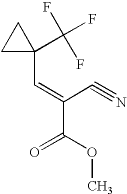

To a solution of Preparation 12 (15.5 g, 66.0 mmol) in anhydrous acetic acid (78 ml) was added dropwise under nitrogen, sodium triacetoxyborohydride (14.0 g, 66.0 mmol) in acetic acid (78 ml). The reaction mixture was then stirred overnight at room temperature. To the reaction mixture was added hydrochloric acid (2N, 250 ml) and the mixture was extracted with dichloromethane (4×250 ml). The combined extracts were washed with brine and concentrated in vacuo. The residue was partitioned between saturated aqueous sodium bicarbonate solution (250 ml) and dichloromethane (250 ml) and adjusted to pH 1 by addition of concentrated hydrochloric acid. The mixture was then extracted with dichloromethane and the combined extracts were concentrated in vacuo to give Preparation 7 (4.6 g). NMR (CDCl3, selected data): 1.6 (m, 2H), 1.8 (s, 2H), 3.9 (s, 3H), 7.9 (s, 1H).

Preparation 8

### Methyl(2)-2-cyano-3-[2-(trifluoromethyl)cyclopropyl]acrylate

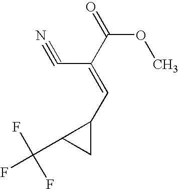

To a solution of pyridinium chlorochromate (4.52 g, 20.95 mmol) in dichloromethane (20 ml) at room temperature was added silica (2.5 g) followed by Preparation 13 (1.85 g, 13.2 mmol) in dichloromethane (10 ml). The reaction mixture was then stirred at room temperature for 2 h before adding diethyl ether (100 ml) filtering through Florisil®, and washing with diethyl ether. The filtrate was concentrated in vacuo to give the intermediate aldehyde (1.4 g).

To a solution of the intermediate aldehyde (860 mg, 6.09 mmol) in acetic acid (1.5 ml) was added methyl cyanoacetate (0.54 ml, 6.12 mmol) via syringe. To this mixture was added piperidine (52 mg, 0.69 mmol) in acetic acid (0.5 ml) and the reaction mixture was stirred overnight at room temperature.

Water (20 ml) was added and the solution was extracted with diethyl ether (3×10 ml). The combined ethereal layers were washed with water (30 ml), saturated aqueous sodium bicarbonate solution (30 ml) and brine (20 ml) before drying (MgSO₄) and concentrating in vacuo. The residue was purified by column chromatography with gradient elution, diethyl ether:pentane [1:3 to 1:1]. The appropriate fractions were concentrated to give Preparation 8 (970 mg, 4.43 mmol).

Preparation 9

### Methyl 2-cyano-3-(1-methylcyclopropyl)acrylate

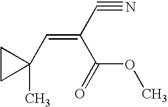

To crude Preparation 10 (19.97 mmol maximum) was added methyl cyanoacetate (1.76 ml, 19.97 mmol) at room temperature. After 15 min, further methyl cyanoacetate (0.5 ml, 5.67 mmol) was added and the reaction mixture was allowed to stand for 45 min.

The reaction mixture was washed with aqueous sodium bicarbonate solution (60 ml) and extracted with dichloromethane (3×50 ml). The combined extracts were dried (MgSO₄), filtered and concentrated in vacuo. To the residue, methyl cyanoacetate (1 ml, 11.33 mmol) was added followed by piperidine (0.1 ml) and the mixture was stirred overnight.

Saturated aqueous sodium bicarbonate solution (75 ml) was added and the aqueous phase was extracted with dichloromethane (3×60 ml). The combined extracts were dried (MgSO₄), filtered and concentrated in vacuo. The crude product was purified by column chromatography (silica) with gradient elution, ethyl acetate:cyclohexane [3:7 to 1:1]. The appropriate fractions were concentrated to give Preparation 9 (380 mg, 2.30 mmol, 11%). NMR (CDCl3, selected data): 1.1 (t, 2H), 1.15 (t, 2H), 1.55 (s, 3H), 3.85 (s, 3H), 7.0 (s, 1H).

Preparation 10

### 1-Methylcyclopropanecarboxaldehyde

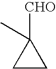

JP 1999-209292; U.S. Pat. No. 6,180,627; EP 997474; J Amer Chem Soc, 1998, 120, 3, 605; U.S. Pat. No. 4,713,477; U.S. Pat. No. 4,754,059.

Preparation 11

### Ethyl 2-[2-cyano-3-methoxy-3-oxoprop-1-enyl]cyclopropanecarboxylate

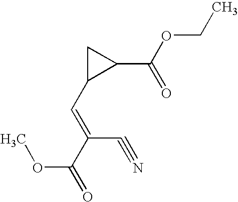

To a solution of ethyl 2-formyl-1-cyclopropanecarboxylate (2 g, 14.0 mmol) in acetic acid (3.5 ml) at room temperature was added methyl cyanoacetate (1.38 g, 1.23 ml, 13.9 mmol) dropwise, via syringe. To this mixture was added piperidine (119 mg, 138 μl, 1.4 mmol) in acetic acid (2.1 ml) and the reaction mixture was stirred at room temperature for 1 h. To the reaction mixture was added water (50 ml) and the mixture was extracted with diethyl ether (3×25 ml). The combined extracts were washed with saturated aqueous sodium hydrogen carbonate solution (30 ml), dried (Na₂SO₄) and concentrated in vacuo. The crude product was purified by column chromatography using an Isolute™ cartridge (silica, 70 g) with gradient elution, cyclohexane:diethyl ether [85:15 to 50:50]. The appropriate fractions were combined and concentrated to give Preparation 11 (1.71 g, 7.67 mmol).

Preparation 12

### Methyl 2-cyano-3-oxo-3-[1-(trifluoromethyl)cyclopropyl]propanoate

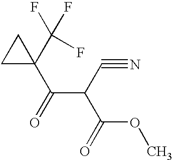

To a solution of methyl cyanoacetate (16.17 g, 163.1 mmol) in acetonitrile (250 ml), under nitrogen and cooled using an ice/acetone bath, was added magnesium chloride (15.61 g, 164.0 mmol). After stirring for 5 min, triethylamine (45.48 ml, 326.3 mmol) was added and the slurry was stirred for a further 1.5 h. To the slurry was added dropwise Preparation 14 (28.0 g, 162.3 mmol) in dichloromethane (40 ml), maintaining the temperature of the reaction mixture at approximately 0° C. To the reaction mixture was added hydrochloric acid (2N, 250 ml), followed by tert-butyl methyl ether (250 ml). The organic layer was separated and the aqueous layer was re-extracted with tert-butyl methyl ether (250 ml). The combined organic phases were then washed with brine and concentrated in vacuo. To the residue was added dichloromethane (500 ml) and the solution was extracted with saturated aqueous sodium hydrogen carbonate solution (2×500 ml). To the combined aqueous extracts was added dichloromethane (300 ml) and the mixture was adjusted to pH 1 by addition of concentrated hydrochloric acid (40 ml). The organic phase was separated, washed with water, dried and concentrated in vacuo to give Preparation 12 (12.7 g) as a red oil. NMR (CDCl3, selected data): 1.3 (m, 2H), 1.5 (m, 2H), 3.9 (m, 3H), 13.5 (s, 1H).

Preparation 13

### 2-(Trifluoromethyl)cyclopropyl]methanol

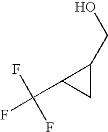

Lithium aluminium hydride (1M in diethyl ether, 13.2 ml, 13.2 mmol) was added to diethyl ether (20 ml) via syringe and the solution was cooled to 0° C. To this solution was added Preparation 15 (2.4 g, 13.2 mmol) in diethyl ether (10 ml), dropwise via syringe before warming to room temperature and leaving overnight. The reaction mixture was cooled to 0° C. and water (0.5 ml) added, followed by aqueous sodium hydroxide solution (1N, 0.5 ml) and further water (1.5 ml). The mixture was warmed to room temperature and filtered through Celite®, washing with diethyl ether (200 ml). The filtrate was then concentrated in vacuo to give Preparation 13 (2.5 g, 17.86 mmol).

Preparation 14

### 1-(Trifluoromethyl)cyclopropanecarbonyl Chloride

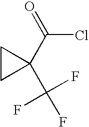

To a solution of Preparation 16 (25.0 g, 162.3 mmol) in anhydrous dichloromethane (250 ml), under nitrogen, was added dropwise N,N-dimethylformamide (15 drops), followed by further dropwise addition of oxalyl chloride (21.2 ml, 243.5 mmol). The reaction mixture was then stirred overnight, under nitrogen, at room temperature before concentrating in vacuo to give Preparation 14 (28.0 g) as an oil.

Preparation 15

### Ethyl 2-(trifluoromethyl)cyclopropanecarboxylate

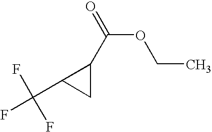

Sodium hydride (60% dispersion in oil, 2.61 g, 65.45 mmol) was washed with hexane (50 ml) and the solvent removed via syringe under nitrogen. To the sodium hydride was added dimethyl sulfoxide (80 ml), followed by trimethylsulfoxonium iodide (14.4 g, 65.45 mmol), added portionwise. The reaction mixture was stirred at room temperature for 2 h and a solution of ethyl 4,4,4-trifluorocrotonate (10 g, 8.88 ml, 59.50 mmol) in dimethyl sulphoxide (40 ml) was added dropwise. The reaction mixture was then stirred at room temperature for 60 h before adding ice/water (250 ml) and extracting with diethyl ether (3×100 ml). The combined ethereal extracts were washed with brine (100 ml), dried (MgSO₄), filtered and concentrated in vacuo. The residue was purified by column chromatography, eluting with diethyl ether/pentane [1:1]. The fractions were then distilled at atmospheric pressure to give Preparation 15 (1.4 g, 7.69 mmol).

Preparation 16

### 1-(Trifluoromethyl)cyclopropanecarboxylic Acid

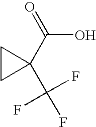

Dmowski, W; Wolniewicz, A; Journal of Fluorine Chemistry 102 (2000) 141-146.

Preparation 17

### 2,6-dichloro-4-(trifluoromethyl)phenyldiazonium Tetrafluoroborate

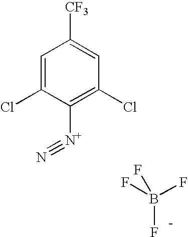

To a stirred solution of 2,6-dichloro-4-(trifluoromethyl)aniline (11.74 g, 0.05 mol) in ethanol (12 ml) at −5° C. was added tetrafluoroboric acid (48% in water, 14 ml, 0.11 mol). A white precipitate formed accompanied by an increase in temperature to 5° C. The reaction mixture was recooled to −5° C. Isoamyl nitrite (6.58 g, 56.1 mmol) was added dropwise over 5 min, the temperature rose to 3° C. The reaction mixture was cooled to −5° C. and stirred for 5 min before allowing to warm to ambient temperature for 30 min. The reaction mixture was filtered and the precipitate washed with absolute ethanol (11 ml) and diethyl ether (10 ml) to give Preparation 17 (14.91 g, 89%) as a crystalline solid.

NMR (D20, selected data): 8.3 (s, 2H).

### EXAMPLES

### Example 1

### 5-amino-1-[2,6-dichloro-4-(trifluoromethyl)phenyl]-4-[1-(trifluoromethyl)cyclopropyl]-1H-pyrazole-3-carbonitrile

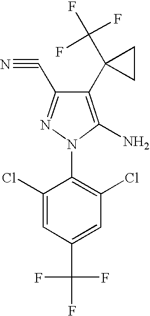

To a solution of tetrafluoroboric acid (54%, 0.54 g, 6.15 mmol) in diethyl ether was added water (1 ml) and 2,6-dichloro-4-(trifluoromethyl)phenylamine (0.5 g, 2.17 mmol). The mixture was stirred for 10 min and then cooled to 0° C. To the mixture was added sodium nitrite (0.15 g, 2.17 mmol) in water (0.3 ml) and the reaction mixture was stirred for 1 h. The solid material was collected by filtration, washed with aqueous tetrafluoroboric acid (2 ml) and water (2 ml) and dried overnight in vacuo to give the diazonium salt.

A solution of Preparation 1 (74 mg, 0.30 mmol) in methanol (1 ml) was adjusted to pH 4 by addition of glacial acetic acid (0.18 g) and cooled to 0° C. To this solution was added the diazonium salt (100 mg, 0.30 mmol) and the reaction mixture was stirred overnight. Sodium carbonate (0.19 g) was added and the solution was filtered, dried and concentrated in vacuo to give the product. NMR (CDCl3, selected data): 1.15 (s, 2H), 1.5 (s, 2H), 4.9 (s, 2H), 7.8 (s, 2H).

Alternative Procedure:

To a solution of Preparation 1 (100 mg, 0.41 mmol) in methanol (1 ml), at 0° C., was added pyridine (0.1 ml, 1.22 mmol) and 4-dimethylaminopyridine (10 mg, 0.08 mmol). The mixture was stirred at 0° C. for 15 min, before addition of Preparation 17 (133 mg, 0.41 mmol). The reaction mixture was then stirred at 0° C. for 3 h. An aliquot of the reaction mixture (0.5 ml) was extracted and concentrated in vacuo. To the residue was added water (5 ml) and the solution was extracted with dichloromethane (3×3 ml). The combined extracts were dried (MgSO₄) and concentrated in vacuo to give an amber oil (51.3 mg). GC-MS analysis indicated the oil contained Example 1 (43.1%, peak area ratio). ¹H nmr confirmed the presence of the compound of Example 1.

Alternative Procedure:

To a solution of Preparation 1 (50 mg, 0.20 mmol) in methanol (0.5 ml), at 0° C., was added pyridine (0.03 ml, 0.30 mmol). The mixture was stirred at 0° C. for 15 min, before addition of Preparation 17 (67 mg, 0.20 mmol). The reaction mixture was then stirred at 0° C. for 3 h, before concentrating in vacuo and adding water (5 ml) to the residue. The resultant solution was extracted with dichloromethane (3×3 ml) and the combined extracts were dried (MgSO₄) and concentrated in vacuo to give an amber oil (73.2 mg). GC-MS analysis indicated the oil contained Example 1 (47.8%, peak area ratio). ¹H nmr confirmed the presence of the compound of Example 1.

Alternative Procedure:

To Preparation 1 (100 mg, 0.41 mmol) was added methanol (1 ml) and the mixture was sonicated for a few minutes. To the solution was added Preparation 17 (170 mg, 0.52 mmol) in methanol (1.3 ml) and sodium hydrogen carbonate (100 mg, 1.19 mmol). The reaction mixture was then stirred at room temperature for 4.5 h. After 4.5 h, a sample removed from the reaction mixture was analysed by GC-MS and showed that Example 1 (15.4%, peak area ratio) was present. After 80 h, GC-MS showed Example 1 (16.4%, peak area ratio) was present. The remaining reaction mixture was heated at 50° C. for 3 h before concentrating in vacuo to give the compound of Example 1 (19.2% peak area ratio) according to GC-MS.

Alternative Procedure:

To Preparation 1 (105 mg, 0.43 mmol) was added methanol (1 ml) and the mixture was sonicated for a few minutes. The solution was cooled using an ice bath and stirred for 15 min. To the solution was added Preparation 17 (180 mg, 0.55 mmol) in methanol (1.5 ml) and sodium acetate (48 mg, 0.59 mmol). The reaction mixture was then stirred at room temperature for 4.5 h. After 4.5 h, a sample was removed from the reaction mixture, analysed by GC-MS and showed the compound of Example 1 (30.7%, peak area ratio) was present.

Alternative Procedure:

To a solution of Preparation 1 (100 mg, 0.41 mmol) in methanol (1 ml) was added acetic acid (0.07 ml, 1.2 mmol). The reaction mixture was cooled to 0° C. and Preparation 17 (134 mg, 0.41 mmol) was added. The reaction mixture was allowed to warm to room temperature and sodium carbonate (129 mg, 1.2 mmol) was added. The reaction mixture was then stirred at room temperature for 2 h. After 2 h, an aliquot removed from the reaction mixture indicated the compound of Example 1 (27.5%, peak area ratio) had formed.

Alternative Procedure:

To a solution of Preparation 1 (100 mg, 0.41 mmol) in methanol (1 ml) was added aqueous potassium carbonate solution (2M, 0.2 ml, 0.4 mmol). The mixture was stirred at room temperature for 30 min and Preparation 17 (134 mg, 0.41 mmol) was added. The reaction mixture was then stirred at room temperature for 24 h. After 2 h, an aliquot removed from the reaction mixture indicated the compound of Example 1 (12.7%, peak area ratio) was present.

### Example 2

### 5-amino-1-[2,6-dichloro-4-(trifluoromethyl)phenyl]-4-[2-(trifluoromethyl)cyclopropyl]-1-1H-pyrazole-3-carbonitrile

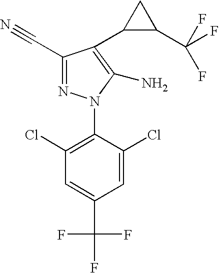

To concentrated sulphuric acid (1.19 g, 12.2 mmol) at 0° C. was added sodium nitrite (168 mg, 2.44 mmol). The mixture was stirred at room temperature for 10 min, before acetic acid (2 ml) was added. To this solution was added 2,6-dichloro-4-(trifluoromethyl)phenylamine (510 mg, 2.24 mmol) in acetic acid (4 ml) via syringe at 0° C. The mixture was warmed to room temperature, with stirring, and then heated at 55° C. for 1 h. After cooling to room temperature, the mixture was added dropwise to Preparation 2 (500 mg, 2.03 mmol) in acetic acid (6 ml). The final reaction mixture was then stirred at room temperature for 1 h.

Water (30 ml) was added followed by dichloromethane (50 ml). The two layer system was separated and the aqueous phase extracted with dichloromethane (2×20). The combined organic layers were then stirred vigorously with concentrated ammonium hydroxide solution (70 ml) and water (20 ml) overnight. The two layers were separated and the organic phase washed with water (100 ml) and brine (50 ml), dried (MgSO₄) and concentrated. The crude product was purified by column chromatography using an Isolute™ cartridge (silica, 10 g) and gradient elution, pentane:diethyl ether [1:0 to 3:1 to 1:1]. The appropriate fractions were concentrated to give the product (169 mg, 0.39 mmol) as a white solid. MS (ES): M/Z [MH+]=429.0, C15H8Cl2F6N4+H requires 429.0. NMR (CDCl3, selected data): 1.4 (m, 1H), 1.5 (m, 1H), 1.85 (m, 1H), 2.05 (m, 1H), 3.65 (s, 2H), 7.75 (s, 2H).

### Example 3

### 5-amino-1-[2,6-dichloro-4-(trifluoromethyl)phenyl]-4-(1-methylcyclopropyl)-1H-pyrazole-3-carbonitrile

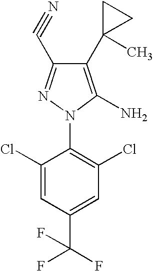

To a cooled solution of sodium nitrite (176 mg, 2.56 mmol) in concentrated sulphuric acid (700 μl) was added glacial acetic acid (2 ml). The mixture was stirred for 15 min and 2,6-dichloro-4-(trifluoromethyl)phenylamine (546 mg, 2.38 mmol) in glacial acetic acid (4 ml) was added dropwise. After stirring for a further 15 min, the reaction mixture was heated at 57° C. for 1 h. Upon cooling to room temperature, the mixture was added carefully to a solution of Preparation 3 (300 mg, 1.83 mmol) in glacial acetic acid (6 ml) and water (10 ml), ensuring the temperature of the reaction mixture did not rise above 14° C. The reaction mixture was then allowed to warm to room temperature and stirred for 2 h.

Water (40 ml) was added and the resulting mixture was extracted with dichloromethane (2×50 ml). The combined extracts were dried (Na₂SO₄) and concentrated in vacuo. To the residue was added water (10 ml) and ammonia (0.88M, 10 ml) and the solution was stirred overnight. To the solution was added dichloromethane (60 ml) and the mixture was washed with water (2×40 ml). The organic layer was separated, dried (Na₂SO₄) and concentrated in vacuo. The crude product was purified by column chromatography (silica) eluting with hexane followed by ethyl acetate and cyclohexane [1:1]. The appropriate fractions were concentrated to give the product (490 mg, 1.31 mmol, 83%). NMR (CDCl3, selected data) 0.7-0.9 (m, 4H), 1.35 (s, 3H), 3.7 (s, 2H), 7.8 (s, 2H).

### Example 4

### 5-amino-1-(6-chloropyridin-3-yl)-4-(1-methylcyclopropyl)-1H-pyrazole-3-carbonitrile

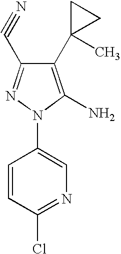

To a solution of Preparation 4 (45 mg, 0.20 mmol) in acetonitrile (2 ml) at 0° C. was added Preparation 3 (25 mg, 0.13 mmol). The reaction mixture was then stirred at 0° C. for 20 min and at room temperature for 3 h. The reaction mixture was concentrated in vacuo and to the residue was added dichloromethane (2 ml) and saturated ammonium hydroxide solution (1.5 ml). The mixture was stirred for 1 h, before water (10 ml) was added and the mixture was extracted with dichloromethane (3×10 ml). The combined extracts were dried (Na₂SO₄) and concentrated in vacuo.

To the residue was added dichloromethane (2 ml), water (2 ml) and ammonia solution (0.88M, 2 ml) and the mixture was stirred vigorously for 5 h. Water (10 ml) was added and the resulting mixture was extracted with dichloromethane (2×10 ml). The combined extracts were dried (Na₂SO₄) and concentrated in vacuo. The crude product was purified by column chromatography (silica) with gradient elution, ethyl acetate:cyclohexane [3:7 to 1:1]. The appropriate fractions were combined and concentrated to give the product (24 mg, 68%) as an orange solid.

NMR (CDCl3, selected data): 1.7-1.9 (m, 4H), 1.3 (s, 3H), 7.5 (d, 1H), 7.9 (dd, 1H), 8.7 (s, 1H).

### Example 5

### 5-amino-1-(2-chloropyridin-3-yl)-4-(1-methylcyclopropyl)-1H-pyrazole-3-carbonitrile

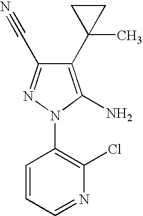

A solution of Preparation 5 (45 mg, 0.20 mmol) and Preparation 3 (25 mg, 0.13 mmol) in dichloromethane (2 ml), concentrated ammonia (2 ml) and water (2 ml) was stirred overnight at room temperature. The reaction mixture was washed with water (10 ml) and extracted with dichloromethane (2×7 ml). The extracts were dried (Na₂SO₄), filtered and concentrated in vacuo. The residue was purified by column chromatography with gradient elution, ethyl acetate:cyclohexane [4:1 to 7:3]. The appropriate fractions were concentrated to give the product (7 mg, 0.03 mmol, 20%). MS (ES): M/Z [MH+]=274.12, C13H12ClN5+H requires 274.1. NMR (CDCl3, selected data): 0.7-0.9 (m, 4H), 1.3 (s, 3H), 3.9 (s, 2H), 7.45 (m, 1H), 7.85 (dd, 1H), 8.55 (m, 1H).

### Example 6

### Ethyl2-{5-amino-3-cyano-1-[2,6-dichloro-4-(trifluoromethyl)phenyl]-1H-pyrazol-4-yl}cyclopropanecarboxylate

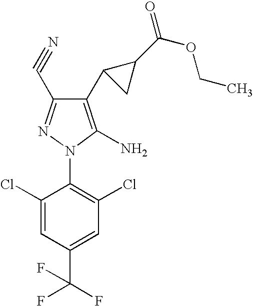

To a stirred solution of 2,6-dichloro-4-(trifluoromethyl)phenylamine (3.0 g, 13 mmol) in ethanol (3 ml) at −5° C. was added tetrafluoroboric acid (48% in water, 27.3 mmol). To the mixture was added isoamyl nitrite (1.8 ml, 13.6 mmol), dropwise over 10 min, and the reaction mixture was stirred for 30 min at room temperature. The solid was filtered and washed with ethanol, followed by diethyl ether, to give the diazonium salt (3.2 g, 9.73 mmol, 75%) as a white solid. To a solution of Preparation 6 (50 mg, 0.20 mmol) in acetonitrile (2 ml) at 0° C. was added diazonium salt (65 mg, 0.20 mmol). The reaction mixture was then allowed to warm to room temperature with stirring. The solution was concentrated under a stream of nitrogen and to the residue was added dichloromethane (2.5 ml), ammonia (0.880, 2.5 ml) and water (2.5 ml), with vigorous stirring.

The mixture was partitioned between water (20 ml) and dichloromethane (20 ml) and the two layers were separated. The aqueous layer was extracted with dichloromethane (2×10 ml) and the combined organic layers were dried (Na₂SO₄) and concentrated in vacuo. The residue was loaded on to an Isolute™ column (silica, 2 g) in a mixture of cyclohexane and dichloromethane mixture (4:1) and eluted with cyclohexane:ethyl acetate [1:0 to 3:7]. The appropriate fractions were combined and concentrated to give the product (15 mg, 0.03 mmol, 17%) as an orange oil. MS (ES): M/Z [MH+]=433.42, C17H13Cl2F3N4O2+H requires 433.04459. NMR (CDCl3, selected data): 1.35 (t, 3H), 1.6-1.7 (m, 2H), 1.9-2.0 (m, 1H), 2.0-2.1 (m, 1H), 3.65 (s, 2H), 4.2 (q, 2H), 8.8 (m, 2H).

Further compounds that can be prepared by the process of the present invention include:

### Example 7

### 5-Amino-3-cyano-4-(2,2-dibromocyclopropyl)-1-(2,6-dichloro-4-trifluoromethylphenyl)pyrazole

The compound is an off-white solid, m.p. 178-179° C. δ (CDCl₃): 2.28 (d, 2H), 2.61 (t, 1H), 3.80 (br.s,2H), 7.8 (s,2H). MS (thermospray): M/Z [M+H] 516.4; C₁₄H₇Br₂Cl₂F₃N₄+H requires 516.84.

### Example 8

### 5-Amino-3-cyano-4-(2,2-dibromocylopropyl)-1-(2,6-dichloro-4-pentafluorothiophenyl)pyrazole

The compound is a white solid, m.p. 178-180° C. δ (CDCl₃): 2.29 (d,2H), 2.60 (t,1H), 3.89 (br.s,2H), 7.93 (d,2H). MS (thermospray): M/Z [M+H] 574.7; C₁₃H₇Br₂Cl₂F₅N₄S+H requires 574.81.

### Example 9

### 5-Amino-3-cyano-4-(2,2-dichlorocyclopropyl)-1-(2,6-dichloro-4-pentafluorothiophenyl)pyrazole

The compound is an off-white solid, m.p. 90-95° C. δ (CDCl₃): 2.23 (m,2H), 2.56 (t,1H), 3.84 (br.s,2H), 7.83 (s,2H). MS (thermospray): M/Z [M+H] 487.3; C₁₃H₇Cl₄F₅N₄S+H requires 486.9.

### Example 10

### 5-Amino-3-cyano-1-(2,6-dichloro-4-trifluoromethylphenyl)-4-(4-methylphenyl)pyrazole

The compound is an off-white crystalline solid, m.p.243-4° C. NMR(CDCl₃): 2.42 (s, 3H), 3.87 (br. s, 2H), 7.33 (d, 2H), 7.44 (d, 2H), 7.82 (s, 2H). MS (thermospray) M/Z [M+H] 411.1; C₁₈H₁₁Cl₂F₃N₄+H requires 411.04.

### Example 11

### 5-Amino-3-cyano-1-(2,6-dichloro-4-trifluoromethylphenyl)-4-phenylpyrazole

The compound is a white solid, m.p. 198-9° C. NMR(CDCl₃): 3.9 (br. s, 2H), 7.4 (m, 1H), 7.52 (m, 4H), 7.82 (s, 2H). MS (thermospray): M/Z [M+H] 397.1; C₁₇H₉Cl₂F₃N₄+H requires 397.02.

### Example 12

### 5-Amino-3-cyano-1-(2,6-dichloro-4-trifluoromethylphenyl)-4-(3-nitrophenyl)pyrazole

The compound is an pale yellow solid, m.p. 212-4° C. NMR(CDCl₃): 4.02 (br. s, 2H), 7.75 (t, 1H), 7.86 (s, 2H), 7.98 (dd, 1H), 8.26 (dd, 1H), 8.41 (dd, 1H). MS (thermospray): M/Z [M+NH₄] 459.2; C₁₇H₈Cl₂F₃N₅O₂+NH₄ requires 459.03.

### Example 13

### 5-Amino-4-(4-bromophenyl)-3-cyano-1-(2,6-dichloro-4-trifluoromethylphenyl)pyrazole

The compound is an off-white solid, m.p. 242-4° C. NMR(CDCl₃): 3.9 (br. s, 2H), 7.43 (d, 2H), 7.64 (d, 2H), 7.82 (s, 2H). MS (thermospray): M/Z [M+H] 474.7; C₁₇H₈BrCl₂F₃N₄+H requires 474.9.

### Example 14

### 5-Amino-4-(4-chlorophenyl)-3-cyano-1-(2,6-dichloro-4-trifluoromethylphenyl)pyrazole

The compound is an off-white solid, m.p. 235-7° C. NMR(CDCl₃): 3.9 (br. s, 2H), 7.5 (s, 4H), 7.82 (s, 2H). MS (thermospray): M/Z [M+H] 430.8; C₁₇H₈Cl₃F₃N₄+H requires 430.98.

### Example 15

### 5-Amino-3-cyano-1-(2,6-dichloro-4-trifluoromethylphenyl)-4-(4-fluorophenyl)pyrazole

The compound is a white solid, m.p. 222-3° C. NMR(CDCl₃): 3.87 (br. s, 2H), 7.22 (m, 2H), 7.54 (m, 2H), 7.82 (s, 2H). MS (thermospray): M/Z [M+H] 415.0; C₁₇H₈Cl₂F₄N₄+H requires 415.01.

### Example 16

### 5-Amino-3-cyano-4-(3,5-dichlorophenyl)-1-(2,6-dichloro-4-trifluoromethylphenyl)pyrazole

The compound is a white solid, m.p. 228-30° C. NMR(CDCl₃): 3.92 (br. s, 2H), 7.38 (d, 1H), 7.43 (d, 2H), 7.82 (s, 2H). MS (thermospray): M/Z [M+H] 464.7; C₁₇H₇Cl₄F₃N₄+H requires 464.9.

### Example 17

### 5-Amino-4-(3-chloro-4-fluorophenyl)-3-cyano-1-(2,6-dichloro-4-trifluoromethylphenyl)pyrazole

The compound is an off-white solid, m.p. 197-8° C. NMR(CDCl₃): 3.9 (br. s, 2H), 7.3 (t, 1H), 7.45 (m, 1H), 7.59 (dd, 1H), 7.82 (s, 2H). MS (thermospray): M/Z [M+H] 448.9; C₁₇H₇Cl₃F₄N₄+H requires 448.98.

### Example 18

### 5-Amino-3-cyano-1-(2,6-dichloro-4-trifluoromethylphenyl)-4-(3-methoxycarbonylphenyl)pyrazole

The compound is a light brown solid, m.p. 214-6° C. NMR(CDCl₃): 3.95 (s+br. s, 5H), 7.6 (t, 1H), 7.79 (d, 1H), 7.80 (s, 2H), 8.05 (d, 1H), 8.09 (s, 1H). MS (thermospray): M/Z [M+NH₄] 472.2; C₁₉H₁₁Cl₂F₃N₄O₂+NH₄ requires 472.06.

### Example 19

### 5-Amino-4-(3-aminophenyl)-3-cyano-1-(2,6-dichloro-4-trifluoromethylphenyl)pyrazole

The compound is a pale brown crystalline solid, m.p. 187° C. NMR(CDCl₃): 2.8 (br. s, 2H), 3.94 (br. s, 2H), 6.7 (d, 1H), 6.87 (s, 1H), 6.89 (d, 1H), 7.28 (dd, 1H), 7.8 (s, 2H). MS (thermospray): M/Z [M+H] 412.1; C₁₇H₁₀Cl₂F₃N₅+H requires 412.03.

### Example 20

### 5-Amino-3-cyano-1-(2,6-dichloro-4-trifluoromethylphenyl)-4-(4-methoxyphenyl)pyrazole

The compound is an off-white crystalline solid, m.p. 222° C. with softening at 192° C. NMR(CDCl₃): 3.88 (s+br. s, 5H), 7.06 (d, 2H), 7.47 (d, 2H), 7.81 (s, 2H). MS (thermospray): M/Z [M+H] 427.4; C₁₈H₁₁Cl₂F₃N₄O+H requires 427.03.

### Example 21

### 5-Amino-3-cyano-1-(2,6-dichloro-4-trifluoromethylphenyl)-4-(3,4-methylenedioxyphenyl)pyrazole

The compound is a pale brown crystalline solid, m.p. 222° C. with softening at 198° C. NMR(CDCl₃): 3.86 (br. s, 2H), 6.03 (s, 2H), 6.86 (d, 1H), 6.9 (m, 2H), 7.81 (s, 2H). MS (thermospray): M/Z [M+H] 440.7; C₁₈H₉Cl₂F₃N₄O₂+H requires 441.01.

### Example 22

### 5-Amino-3-cyano-1-(2,6-dichloro-4-trifluoromethylphenyl)-4-(3,4-dimethoxyphenyl)pyrazole

The compound is a pale pink crystalline solid, m.p. 250° C. NMR(CDCl₃): 3.88 (br. s, 2H), 3.94 (s, 3H), 3.97 (s, 3H), 6.99 (d, 1H), 7.01 (m, 2H), 7.81 (s, 2H). MS (thermospray): M/Z [M+H] 457.0; C₁₉H₁₃Cl₂F₃N₄O₂+H requires 457.05.

### Example 23

### 5-Amino-3-cyano-1-(2,6-dichloro-4-trifluoromethylphenyl)-4-(3-fluorophenyl)pyrazole

The compound is a white crystalline solid, m.p. 164° C. NMR(CDCl₃): 3.96 (br. s, 2H), 7.04 (m, 1H), 7.13 (m, 1H), 7.18 (m, 1H), 7.24 (m, 1H), 7.81 (s, 2H). MS (thermospray): M/Z [M+H] 415.0; C₁₇H₈Cl₂F₄N₄+H requires 415.01.

### Example 24

### 5-Amino-4-(3-chlorophenyl)-3-cyano-1-(2,6-dichloro-4-trifluoromethylphenyl)pyrazole

The compound is a white crystalline solid, m.p. 161-2° C. NMR(CDCl₃): 3.94 (br.s, 2H), 7.38 (m, 1H), 7.47 (m, 2H), 7.51 (m, 1H), 7.81 (s, 2H). MS (thermospray): M/Z [M+H] 431.1; C₁₇H₈Cl₃F₃N₄+H requires 430.98.

### Example 25

### 5-Amino-3-cyano-1-(2,6-dichloro-4-trifluoromethylphenyl)-4-(2-fluorophenyl)pyrazole

The compound is a white crystalline solid, m.p. 197° C. NMR(CDCl₃): 3.9 (br. s, 2H), 7.18 (m, 2H), 7.4 (m, 1H), 7.6 (m, 1H), 7.81 (s, 2H). MS (thermospray): M/Z [M+H] 415.0; C₁₇H₈Cl₂F₄N₄+H requires 415.01.

### Example 26

### 5-Amino-3-cyano-1-(2,6-dichloro-4-trifluoromethylphenyl)-4-(2-methoxyphenyl)pyrazole

The compound is a white crystalline solid, m.p. 193° C. NMR(CDCl₃): 3.89 (s, 3H), 3.91 (br. s, 2H), 7.0 (d, 1H), 7.09 (t, 1H), 7.37 (dd, 1H), 7.5 (d, 1H), 7.78 (s, 2H).

MS (thermospray): M/Z [M+H] 427.0; C₁₈H₁₁Cl₂F₃N₄O+H requires 427.03.

### Example 27

### 5-Amino-3-cyano-1-(2,6-dichloro-4-trifluoromethylphenyl)-4-(2-methylphenyl)pyrazole

The compound is a pale brown crystalline solid, m.p. 199-202° C. NMR(CDCl₃): 2.32 (s, 3H), 3.62 (br. s, 2H), 7.33 (m, 4H), 7.81 (s, 2H). MS (thermospray): M/Z [M+H] 411.0; C₁₈H₁₁Cl₂F₃N₄+H requires 411.04.

### Example 28

### 5-Amino-3-cyano-1-(2,6-dichloro-4-trifluoromethylphenyl)-4-(2-trifluoromethylphenyl)pyrazole

The compound is a white crystalline solid, m.p. 210-2° C. NMR(CDCl₃): 3.57 (br. s, 2H), 7.49 (d, 1H), 7.6 (m, 1H), 7.69 (m, 1H), 7.81 (s, 2H), 7.83 (m, 1H). MS (thermospray): M/Z [M+H] 465.0; C₁₈H₈Cl₂F₆N₄+H requires 465.01.

### Example 29

### 5-Amino-4-(2-chlorophenyl)-3-cyano-1-(2,6-dichloro-4-trifluoromethylphenyl)pyrazole

The compound is a pale brown crystalline solid, m.p. 192-3° C. NMR(CDCl₃): 3.76 (br. s, 2H), 7.4 (m, 2H), 7.52 (m, 2H), 7.8 (s, 2H). MS (thermospray): M/Z [M+H] 431.2; C₁₇H₈Cl₃F₃N₄+H requires 430.98.

### Example 30

### 5-Amino-3-cyano-1-(2,6-dichloro-4-trifluoromethylphenyl)-4-(1-naphthyl)pyrazole

The compound is an off-white crystalline solid, m.p. 208° C. NMR(CDCl₃): 3.65 (br. s, 2H), 7.6 (m, 4H), 7.75 (m, 1H), 7.85 (m, 2H), 7.97 (m, 2H). MS (thermospray): M/Z [M+H] 447.0; C₂₁H₁₁Cl₂F₃N₄+H requires 447.04.

### Example 31

### 5-Amino-3-cyano-1-(2,6-dichloro-4-trifluoromethylphenyl)-4-(3-pyridyl)pyrazole

The compound is an off-white solid, m.p. 265-7° C. NMR(CDCl₃): 4.38 (br. s, 2H), 7.55 (m, 1H), 7.82 (s, 2H), 8.07 (d, 1H), 8.59 (m, 1H), 8.92 (s, 1H). MS (thermospray): M/Z [M+H] 398.2; C₁₆H₈Cl₂F₃N₅+H requires 398.01.

### Example 32

### 5-Amino-3-cyano-1-(2,6-dichloro-4-trifluoromethylphenyl)-4-(4-pyridyl)pyrazole

The compound is a white solid, m.p. 266-8° C. (decomp.). NMR(CDCl₃): 4.1 (br.s, 2H), 7.52 (d, 2H), 7.82 (s, 2H), 8.71 (d, 2H). MS (thermospray): M/Z [M+H] 397.9; C₁₆H₈Cl₂F₃N₅+H requires 398.01.

### Example 33

### 5-Amino-3-cyano-1-(2,6-dichloro-4-trifluoromethylphenyl)-4-(2-thienyl)pyrazole

The compound is a white solid. A sample recrystallised from methanol had m.p. 206-7° C. NMR(CDCl₃): 4.01 (br. s, 2H), 7.18 (m, 1H), 7.38 (m, 2H), 7.79 (s, 2H). MS (thermospray): M/Z [M+H] 403.2; C₁₅H₇Cl₂F₃N₄S+H requires 402.98.

### Example 34

### 5-Amino-3-cyano-1-(2,6-dichloro-4-trifluoromethylphenyl)-4-(3-thienyl)pyrazole

The compound is a white crystalline solid, m.p. 210-2° C. NMR(CDCl₃): 3.9 (br. s, 2H), 7.41 (m, 1H), 7.5 (m, 2H), 7.81 (s, 2H). MS (thermospray): M/Z [M+H] 403.3; C₁₅H₇Cl₂F₃N₄S+H requires 402.98.

### Example 35

### 5-Amino-3-cyano-1-(2,6-dichloro-4-trifluoromethylphenyl)-4-(2-furanyl)pyrazole

The compound is a very light pink crystalline solid 195-6° C. NMR(CDCl₃): 4.46 (br. s, 2H), 6.52 (d, 1H), 6.78 (d, 1H), 7.43 (s, 1H), 7.86(s, 2H). MS (thermospray): M/Z [M+H] 387.1; C₁₅H₇Cl₂F₃N₄O+H requires 387.0.

### Example 36

### 5-Amino-3-cyano-1-(2,6-dichloro-4-trifluoromethylphenyl)-4-(3-furanyl)pyrazole

The compound is a white solid, m.p. 180-1° C. NMR(CDCl₃): 3.79 (br. s, 2H), 6.79 (s, 1H), 7.6 (m, 1H), 7.78 (m, 1H), 7.82(s, 2H). MS (thermospray): M/Z [M+H] 386.9; C₁₅H₇Cl₂F₃N₄O+H requires 387.0.

### Example 37

### 5-Amino-3-cyano-1-(2,6-dichloro-4-methylphenyl)-4-(2-furanyl)pyrazole

The compound as a white solid, m.p. 191.5-192.5° C. NMR(CDCl₃): 2.43 (s, 3H), 4.43 (br. s, 2H), 6.53 (m, 1H), 6.79 (m, 1H), 7.32 (s, 2H), 7.47 (s, 1H). Microanalysis: Found C, 53.79, H, 2.87, N, 16.65%; C₁₅H₁₀Cl₂F₃N₄O requires C, 54.07, H, 3.03, N, 16.82%.

### Example 38

### 5-Amino-3-cyano-1-(2,6-dichlorophenyl)-4-(2-furanyl)pyrazole

The compound is a light brown solid, m.p. 222.6° C. NMR(CDCl₃): 4.42 (br. s, 2H), 6.52 (m, 1H), 6.78 (m, 1H), 7.5 (m, 4H). MS (thermospray): M/Z [M+H] 318.8; C₁₄H₈Cl₂N₄O+H requires 319.01.

### Example 39

### 5-Amino-4-(2-n-butylphenyl)-3-cyano-1-(2,6-dichlorophenyl-4-trifluoromethylphenyl)pyrazole

The compound is a white solid, m.p. 117.1-117.7° C. NMR(CDCl₃): 0.87 (t, 3H), 1.25 (m, 2H), 1.46 (m, 2H), 2.6 (m, 2H), 3.6 (br. s, 2H), 7.39 (m, 2H), 7.47 (m, 2H), 7.81 (s, 2H). MS (thermospray): M/Z [M+H] 453.0; C₂₁H₁₇Cl₂F₃N₄+H requires 453.09.

### Example 40

### 5-Amino-3-cyano-4-(2,3-dichlorophenyl)-1-(2,6-dichlorophenyl-4-trifluoromethylphenyl)pyrazole

The compound is a white solid, m.p. 201-202° C. NMR(CDCl₃): 3.79 (br. s, 2H), 7.38 (dd, 1H), 7.44 (d, 1H), 7.58 (d, 1H), 7.82 (s, 2H). Microanalysis: Found C, 43.66, H, 1.51, N, 11.97%; C₁₇H₇Cl₄F₃N₄ requires C, 43.81, H, 1.51, N, 12.02%.

### Example 41

### 5-Amino-4-(3-bromoisoxazol-5-yl)-3-cyano-1-(2,6-dichloro-4-trifluoromethylphenyl)pyrazole

The compound is a white crystalline solid, m.p. 227° C. NMR(CDCl₃): 4.86 (br. s, 2H), 6.78 (s, 1H), 7.83 (s, 2H). MS (thermospray): M/Z [M+NH₄] 482.8; C₁₄H₅BrCl₂F₃N₅O+NH₄ requires 482.94.

### Example 42

### Amino-3-cyano-1-(2,6-dichloro-4-trifluoromethylphenyl)-4-(2-methylthiazol-4-yl)pyrazole

The compound is a white solid, m.p. 226-8° C. NMR(d₆-dmso): 2.72 (s, 3H), 6.6 (br. s, 2H), 7.53 (s, 1H), 7.8 (s, 2H). MS (thermospray): M/Z [M+H] 418.2; C₁₅H₈Cl₂F₃N₅S+H requires 417.99.

### Example 43

### 5-Amino-4-(5-bromothien-2-yl)-3-cyano-1-(2,6-dichloro-4-trifluoromethylphenyl)pyrazole

The compound is an off-white solid, m.p. 214° C. NMR(CDCl₃): 3.99 (br. s, 2H), 7.1 (m, 2H), 7.8 (s, 2H). MS (thermospray): M/Z [M+NH₄] 498.0; C₁₅H₆BrCl₂F₃N₄S+NH₄ requires 497.92.

### Example 44

### 5-Amino-3-cyano-1-(2,6-dichloro-4-trifluoromethylphenyl)-4-(5-trifluoromethylsulphenylthien-2-yl)pyrazole

The compound is a white solid, m.p. 162-3° C. NMR(CDCl₃): 4.12 (br. s, 2H), 7.39 (d, 1H), 7.46 (d, 1H), 7.8 (s, 2H). MS (thermospray): M/Z[M+H] 502.9; C₁₆H₆Cl₂F₆N₄S₂+H requires 502.94.

### Example 45

### 5-Amino-3-cyano-4-(3,4-dibromoisoxazol-5-yl)-1-(2,6-dichloro-4-trifluoromethylphenyl)pyrazole

The compound is a white solid, m.p. 279° C. NMR(CDCl₃): 4.49 (br. s, 2H), 7.82 (s, 2H). MS (thermospray): M/Z [M+NH₄] 561.0; C₁₄H₄Br₂Cl₂F₃N₅O+NH₄ requires 560.85.

### Example 46

### 5-Amino-4-(2-chlorofuran-3-yl)-3-cyano-1-(2,6-dichloro-4-rifluoromethylphenyl)pyrazole

The compound is a white solid, m.p. 153° C. NMR(CDCl₃): 4.86 (br. s, 2H), 6.73 (d, 1H), 7.49 (d, 1H), 7.82 (s, 2H). MS (thermospray): M/Z [M+H] 421.0; C₁₅H₆Cl₃F₃N₄O+H requires 420.96.

### Example 47

### 5-Amino-4-(2-bromofuran-3-yl)-3-cyano-1-(2,6-dichloro-4-trifluoromethylphenyl)pyrazole

The compound is a white solid. NMR(d₆-dmso): 6.18 (br. s, 2H), 6.74 (d, 1H), 7.93 (d, 1H), 8.26 (s, 2H). MS (thermospray): M/Z [M+H] 464.3; C₁₅H₆BrCl₂F₃N₄O+H requires 464.91.

### Example 48

### 5-Amino-3-cyano-1-(2,6-dichloro-4-trifluoromethylphenyl)-4-(2-trifluoromethylthiofuran-3-yl)pyrazole

The compound is a white solid, m.p.162° C. NMR(CDCl3): 3.96 (br. s, 2H), 6.85 (d, 1H), 7.82 (d, 1H), 7.82 (s, 2H). MS (thermospray): M/Z [M+NH₄] 503.6; C₁₆H₆Cl₂F₆N₄OS+NH₄ requires 503.98.

### Example 49

### 5-Amino-3-cyano-1-(2,6-dichloro-4-trifluoromethylphenyl)-4-(2-trifluoromethylsulphinylfuran-3-yl)pyrazole

The compound is a white solid, m.p.141-142° C. NMR(CDCl₃): 5.64 (br. s, 2H), 6.65 (d, 1H), 7.8 (d, 1H), 7.82 (s, 2H). MS (thermospray): M/Z [M+H] 502.8; C₁₆H₆Cl₂F₆N₄O₂S+H requires 502.96.

### Example 50A and 50B

### 5-Amino-3-cyano-1-(2,6-dichloro-4-trifluoromethylphenyl)-4-(2-trifluoromethylfuran-3-yl)pyrazole and 5-amino-3-cyano-1-(2,6-dichloro-4-trifluoromethylphenyl)-4-(5-trifluoromethylfuran-3-yl)pyrazole

Combination and evaporation of suitable fractions following chromatography of the reaction mixture gave 5-amino-3-cyano-1-1(2,6-dichloro-4-trifluoromethylphenyl)-4-(2-trifluoromethylfuran-3-yl)pyrazole as a white solid, m.p. 155-7° C. NMR(CDCl₃): 3.79 (br. s, 2H), 6.70 (d, 1H), 7.70 (d, 1H), 7.82 (s, 2H). MS (thermospray): M/Z [M+H] 454.8; C₁₆H₆Cl₂F₆N₄O+H requires 455.0.

The residue obtained by evaporation of fractions containing longer retained materials was further purified to give 5-amino-3-cyano-1-(2,6-dichloro-4-trifluoromethylphenyl)-4-(5-trifluoromethylfuran-3-yl)pyrazole as an amorphous pale brown solid. NMR(d₆-dmso): 6.55 (q, 1H), 6.64 (s, 1H), 7.1 (br. s, 2H), 8.28 (s, 2H). MS (thermospray): M/Z [M+H] 471.0; C₁₆H₆Cl₂F₆N₄O+H requires 472.02.

### Example 51

### 5-Amino-3-cyano-1-(2,6-dichloro-4-trifluoromethylphenyl)-4-(5-trifluoromethylfuran-2-yl)pyrazole

The compound is a pale yellow solid, m.p. 148-151° C. NMR(CDCl₃): 3.53 (br. s, 2H), 6.87 (d, 1H), 6.96 (d, 1H), 7.82 (s, 2H). MS (thermospray): M/Z [M+H] 455.1; C₁₆H₆Cl₂F₆N₄O+H requires 455.0.

### Example 52

### 5-Amino-3-cyano-4-(2,5-dichlorofuran-3-yl)-1-(2,6-dichloro-4-trifluoromethylphenyl)pyrazole

The compound is a white solid, m.p. 193-194° C. NMR(CDCl₃): 3.9 (br. s, 2H), 6.52 (d, 1H), 7.8 (s, 2H). MS (thermospray): M/Z [M+H] 454.7; C₁₅H₅Cl₄F₃N₄O+H requires 454.

1. A process for the preparation of a compound of formula (I) 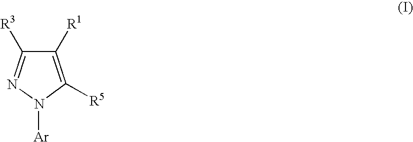
said process comprising the step of reacting a compound of formula (II) 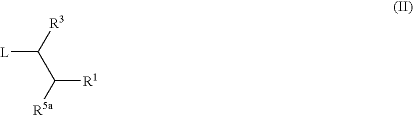
with a compound of formula (III)
AR—N≡N⁺X⁻ (III)
optionally in the presence of an acid, wherein:
Ar is phenyl or pyridyl, optionally independently substituted by 1 to 4 groups selected from the group comprising: C₁₋₄ alkyl, C₁₋₄ alkoxy, C₁₋₄ alkylthio, C₁₋₄ alkylsulphinyl, and C₁₋₄ alkylsulphonyl wherein each of these optional substituent groups may itself be substituted by one or more halogen atoms selected independently; halogen; pentafluorosulfur; and —COOC₁₋₈ alkyl
R¹ is C₁₋₈ alkyl, C₂₋₈ alkenyl provided that said alkenyl is not conjugated with the double bond shown in formula (IV), C₄₋₈ cycloalkyl, C₁₋₈ alkyl(C₃₋₈ cycloalkyl), a 5- or 6-membered heterocycle wihich may be saturated, partially or fully unsaturated designated “het” containing 1, 2 or 3 heteroatoms, which are independently selected from 1, 2 or 3 N atoms, 1 or 2 O atoms and 1 or 2 S atoms, where the valence allows, C₁₋₈ alkylhet, phenyl, C₁₋₈ alkylphenyl; wherein each of the preceding groups may be optionally independently substituted by 1 to 4 groups selected from the group comprising: halogen, C₁₋₄ alkyl, C₁₋₄ alkoxy, C₁₋₄ alkylthio, and —COOC₁₋₈ alkyl; wherein each of these preceding optional substituent groups may be substituted where possible by one or more halogen atoms selected independently; or
R¹ is a group of formula (A): 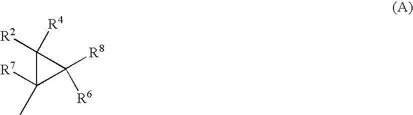
wherein R² and R⁴ are each independently selected from hydrogen, C₁₋₄ alkyl, fluoro, chloro and bromo, or, together with the carbon atom to which they are attached, form a C₃₋₆ cycloalkyl group;
R⁶ and R⁸ are each independently selected from hydrogen, C₁₋₄ alkyl, fluoro, chloro and bromo;
or when R² and R⁴ do not form part of a cycloalkyl group, R² and R⁶, together with the carbon atoms to which they are attrached, may form a C₅₋₇ cyclalkyl group;
R⁷ is hydrogen, C₁₋₄ alkyl optionally substituted with one or more halo, or C₁₋₄ alkoxy;
or R¹ is a fused bicyclic moiety “AB” where the “A” ring is as defined as ‘het’ above and the “B” ring fused thereto in “AB” is a 5- or 6-membered saturated or partially or fully unsaturated carbocycle, or saturated or partially or fully unsaturated heterocycle where the valence allows, which heterocycle contains 1, 2, 3 or 4 hetero-atoms independently selected from 1, 2, 3 or 4 N atoms, 1 or 2 O atoms and 1 or 2 S atoms, where the valence allkows, said R¹ group being linked via the “A” ring to the 4-position of the pyrazole via a carbon-carbon bond, and said R¹ group being optionally substituted by one or more substituents independently selected from halogen, C₁₋₆ alkyl optionally substituted by one or more halogen atoms, C₁₋₆ alkoxy optionally substituted by one or more halogen atoms, C₁₋₆ alkoxycarbonyl optionally substituted by one or more halogen atoms, NO₂, NH₂, CN or S(O)ₘ(C₁₋₆ alkyl optionally substituted by one or more halogen atoms) where m is 0, 1 or 2;
R³ is selected from the group comprising: CN, CF₃, CHO, COR and COOR wherein R is C₁₋₆ alkyl optionally substituted by one or more halogen atoms which may be the same or different;
R⁵ is selected from the group comprising: hydrogen, C₁₋₆ alkyl optionally substituted by one or more halogen atoms which may be the same or different, OH and NH₂;
R^{5a} is selected from the group comprising: C\N, COOH, CHO, COR and COOR wherein R is C₁₋₆ alkyl optionally substituted by one or more halogen atoms which may be the same or different;
L is an activating group; and
X— is a compatible counter ion,
followed by removal of group L.

2. A process for the preparation of a compound of formula (II) in which R^{5a} is CN 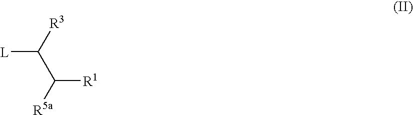
the process comprising treating a compound of formula (IV) 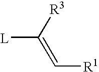
(IV)
with a source of cyanide ions, wherein L, R¹, and R³ are as defined in claim 1.

3. A process for the preparation of a compound of formula (II) 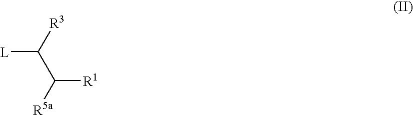
in which R^{5a} is CN, COOH, CHO, COR, and COOR wherein R is C₁₋₆ alkyl optionally substituted by one or more halogen atoms which may be the same or different, the process comprising reacting a compound of formula LCH₂R³ with a base and then reacting the resulting mixture with R^{5a}CH(X)R¹ where X is Cl, Br, I, C₁₋₈ alkylsulphonate or arylsulphonate at room temperature under an inert atmosphere, wherein L, R¹ and R³ are as defined in claim 1.

4. A process for preparing a compound of formula (I) in which R⁵ is NH₂ 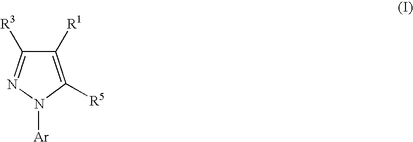
said process comprising a first step of reacting a compound of formula (IV) 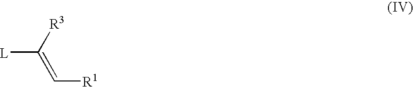
with a source of cyanide ions to produce a compound of formula (II) and subsequently treating the resulting mixture with a compound of formula (III)
Ar—N≡N⁺X⁻ (III)
wherein Ar, L, R¹ R³ and X are as defined in claim 1.

5. A process for preparing a compound of formula (IV), the process comprising the step of reducing a compound of formula (V) 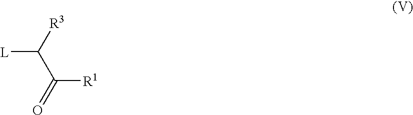
with a complex metal hydride in the presence of acid, wherein L, R¹ and R³ are as defined in claim 1.

6. A compound of formula (II) 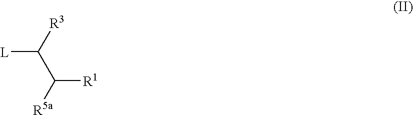
wherein L, R¹, R³ and R^{5a} are as defined in claim 1.

7. A compound of formula (IV) 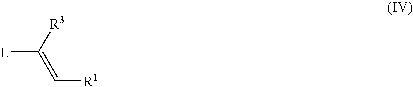
wherein L, R¹ and R³ are as defined in claim 1.

8. A compound of formula (V) 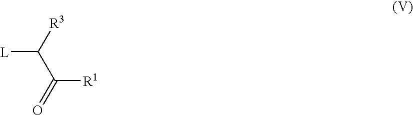
wherein L, R¹ and R³ are as defined in claim 1.

9. A compound of formula (IX) 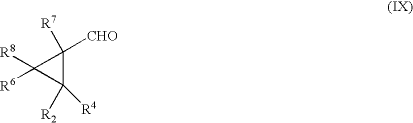
wherein R², R⁴, R⁶, R⁷ and R⁸ are as defined in claim 1.

10. A process or compound as claimed in any of claims 1 to 9, wherein L, when present, is a group selected from: —S(O)ₚR⁹ where p is 1 or 2, R⁹(O)₂PO, COOR⁹ and —COR¹⁰,
wherein R⁹ is selected from: C₁₋₈ alkyl, C₃₋₈ cycloalkyl, (CH₂)ₙPh and (CH₂)ₙ heteroaryl wherein n=0, 1 or 2, each of which groups may be optionally substituted on any carbon atom by one or more groups selected independently from: halogen, hydroxy, cyano, nitro, C₁₋₄ alkoxy, C₁₋₄ haloalkoxy, C₁₋₄ alkanoyl, C₁₋₄ haloalkanoyl, C₁₋₄ alkylsulphinyl, C₁₋₄ haloalkylsulphinyl, C₁₋₄ alkylsulphonyl, C₁₋₄ haloalkylsulphonyl, C₃₋₈ cycloalkyl and C₃₋₈ halocycloalkyl; and R⁹ can be hydrogen; and
wherein R¹⁰ is selected from: C₁₋₈ alkyl, di-C₁₋₈ alkylamino, C₁₋₈ alkylthio, C₃₋₈ cycloalkyl, (CH₂)ₙPh and (CH₂)ₙ heteroaryl wherein n=0, 1 or 2, each of which groups may be optionally substituted on any carbon atom by one or more groups selected independently from: halogen, hydroxy, cyano, nitro, C₁₋₄ alkoxy, C₁₋₄ haloalkoxy, C₁₋₄ alkanoyl, C₁₋₄ haloalkanoyl, C₁₋₄ alkylsulphinyl, C₁₋₄ haloalkylsulphinyl, C₁₋₄ alkylsulphonyl, C₁₋₄ haloalkylsulphonyl, C₃₋₈ cycloalkyl and C₃₋₈ halocycloalkyl; and R¹⁰ can be hydrogen.

11. A process or compound as claimed in any of claims 1 to 10, wherein Ar, when present, is tri-substituted, and more preferably it is substituted at the 2-, 4-, and 6-positions with an optional substituent selected from the group comprising: halogen, C₁₋₄ alkyl, C₁₋₄ alkoxy, C₁₋₄ aalkylthio, SF₅ and —COOC₁₋₈ alkyl, wherein each of these optional substituent groups may itself be submitted where chemically possible by one to three halogen atoms selected independently.

12. A process or compound as claimed in claim 11, wherein Ar, when present, is a phenyl group which bears substituents at the 2-, 4-, and 6-positions, the substituents at those positions, the substituents at those positions being independently selected from chloro, trifluoromethyl, trifluoromethoxy, and pentafluorosulfur.

13. A process or compound as claimed in any of claims 1 to 12, wherein R¹, when present, is selected from: C₁₋₈ alkyl, C₄₋₈ cycloalkyl, a group of formula (A) where A is as defined above in claim 1, a 5- or 6-membered heterocycle which may be saturated or unsaturated designated ‘het’, C₁₋₈ alkylhet, phenyl, and C₁₋₈ alkylphenyl, wherein each of the preceding groups may be optionally independently substituted by 1 to 4 groups selected from the group comprising: halogen, C₁₋₄ alkyl, C₁₋₄ alkoxy, C₁₋₄ thioalkoxy, and —COOC₁₋₈ alkyl, wherein each of these optional substituent groups may itself be substituted where possible by one or more halogen atoms selected independently.

14. A process or compound as claimed in claim 13, wherein R¹ is selected from: C₁₋₈ alkyl, C₄₋₈ cycloalkyl, a group of formula (A) where A is as defined above in claim 1, or a 5- or 6-membered heterocycle which may be saturated or unsaturated designated ‘het’, or C₁₋₈ alkylhet, wherein each of the preceding groups may be optionally independently substituted by 1 to 4 groups selected from the group comprising: halogen, C₁₋₄ alkyl, C₁₋₄ alkoxy, C₁₋₄ thioalkoxy, and —COOC₁₋₈ alkyl, wherein each of these optional substituent groups may itself be substituted where possible by one or more halogen atoms selected independently.

15. A process or compound as claimed in any of claims 1 to 14, wherein R³, when present, is cyano.

16. A process or compound as claimed in any of claims 1 to 15, wherein R⁵, when present, is amino.
